(19) United States
(12) Patent Application Publication
(10) Pub. No.: US 2022/0054687 A1
(43) Pub. Date: Feb. 24, 2022

(54) SYSTEM AND METHOD FOR MITIGATING AIRBORNE CONTAMINATION IN CONDITIONED INDOOR ENVIRONMENTS

(71) Applicants: ARIZONA BOARD OF REGENTS ON BEHALF OF ARIZONA STATE UNIVERSITY, SCOTTSDALE, AZ (US); MAYO FOUNDATION FOR MEDICAL EDUCATION AND RESEARCH, ROCHESTER, MN (US)

(72) Inventors: Erica Forzani, Mesa, AZ (US); Gabriel Pyznar, Tempe, AZ (US); Bhavesh Patel, Scottsdale, AZ (US); Kelly McKay, Southern Pines, NC (US)

(21) Appl. No.: 17/406,675

(22) Filed: Aug. 19, 2021

Related U.S. Application Data
(60) Provisional application No. 63/067,696, filed on Aug. 19, 2020.

(51) Int. Cl. A61L 2/24; A61L 2/02; A61L 2/10; A61L 2/18; A61L 2/20; A61L 2/23; A61L 2/28

(52) U.S. Cl. CPC A61L 2/24; A61L 2/022; A61L 2/10; A61L 2/18; A61L 2/202; A61L 2/23; A61L 2209/22; A61L 2209/111; A61L 2209/13; A61L 2209/14; A61L 2209/16; A61L 2209/212; A61L 2/28

(57) ABSTRACT

A system and method for mitigating airborne contamination in a conditioned indoor environment utilizes one or more sensing modules configured to detect presence and/or concentration of particles and/or aerosols at different locations. A control module employs an artificial intelligence algorithm to selectively activate at least one mitigation module utilizing machine learning programmed rules and output signals from the sensing module(s). The mitigation module(s) are configured to take one or more actions to reduce presence and/or concentration of particles and/or aerosols in the conditioned indoor environment.

### CROSS-REFERENCED TO RELATED APPLICATION(S)

This application claims priority to U.S. Provisional Patent Application No. 63/067,696 filed on Aug. 19, 2020, wherein the entire contents of the foregoing application are hereby incorporated by reference herein.

### BACKGROUND

Airborne diseases are transmitted by the spread of microorganisms (also referred to as microbes) mainly through aerosols and micro droplets. Contaminated micro droplets are frequently generated by an infected host through sneezing, coughing, breathing, speaking, and sweating. Airborne diseases not only affect human health but also detrimentally impact the global economy. Although most efforts are targeted towards protecting individuals from getting infected (e.g., using of personal protective equipment (PPE)), it is also important to promote the maintenance of clean and controlled indoor environments to mitigate airborne contamination and reduce the spread of communicable airborne diseases.

Aerosols are microscopic particles of 0.01 μm to 100 μm in size suspended in air. Ninety-nine percent of aerosols produced by humans (regardless of age, sex, weight, and height) are less than 10 μm. The small size of most aerosols produced by humans is concerning, since smaller aerosols take longer to settle than larger ones and are therefore more likely to be inhaled into the lungs of other individuals. In a turbulent atmosphere, aerosols of 100 μm take an average of 5.8 seconds to settle on surfaces, while 0.5 μm aerosols may take 41 hours to settle. If aerosols contain viable pathogens, they can be a threat while airborne and even after they settle on surfaces since they can generate elements that are sources of contamination. In case of SARS-CoV-2, viruses can be viable on a surface for up to two days.

Although ventilating spaces with fresh air may reduce the concentration of aerosols in indoor environments, introducing large amounts of fresh air may render it difficult to maintain comfortable conditions (e.g., with respect to temperature, humidity, etc.) without expending undue amounts of energy that would increase operating costs and may lead to concomitantly increased carbon emissions.

The art continues to seek improvement in systems and methods for mitigating airborne contamination in conditioned indoor environments, particularly in a manner that does not involve undue expenditure of energy.

### SUMMARY

The present disclosure relates to a system and method for mitigating airborne contamination in a conditioned indoor environment. Multiple sensing modules are configured to detect presence and/or concentration of particles and/or aerosols at different locations. A control module employing an artificial intelligence algorithm configured to selectively activate at least one mitigation module based on utilization of machine learning programmed rules and output signals from one or more sensing modules. The at least one mitigation module is configured to take one or more actions to reduce presence and/or concentration of particles and/or aerosols in the conditioned indoor environment.

In one aspect, the disclosure relates to a system for mitigating airborne contamination in a conditioned indoor environment. The system comprises: at least one sensing module that comprises an aerosol and/or particulate detector configured to detect presence and/or concentration of particles and/or aerosols at a location in the conditioned indoor environment; at least one mitigation module configured to take one or more actions to reduce presence and/or concentration of particles and/or aerosols in the conditioned indoor environment; and a control module employing an artificial intelligence algorithm configured to selectively activate the at least one mitigation module based on utilization of machine learning programmed rules and output signals from one or more sensing modules of the at least one sensing module.

In certain embodiments, the system further comprises at least one sampling module configured to automatically collect an air sample from the conditioned indoor environment based on utilization of machine learning programmed rules and output signals from one or more modules of the at least one sensing module.

In certain embodiments, the at least one sampling module comprises at least one of a chemical analyzer or a biological analyzer configured to identify one or more constituents of the air sample.

In certain embodiments, the at least one sensing module comprises at least one of a temperature sensor, a pressure sensor, a carbon dioxide sensor, and a humidity sensor.

In certain embodiments, each sensing module of the at least one sensing module comprises an occupancy sensor configured to sense the presence of at least one human within the conditioned indoor environment.

In certain embodiments, the at least one mitigation module comprises a ventilation module configured to increase exchange of air between the conditioned indoor environment and an outdoor environment, wherein the ventilation module comprises an inlet fan and an outlet fan.

In certain embodiments, the at least one mitigation module comprises at least one of a wet scrubber or a dry scrubber.

In certain embodiments, the at least one mitigation module comprises a disinfection module configured to disinfect air of the conditioned indoor environment.

In certain embodiments, the disinfection module comprises at least one of an ozone generator or an ultraviolet lamp.

In certain embodiments, the at least one mitigation module comprises a filtration module.

In certain embodiments, the at least one mitigation module comprises a plurality of mitigation modules of different types.

In certain embodiments, at least a portion of the at least one mitigation module is positioned in ductwork of an HVAC apparatus associated with the conditioned indoor environment.

In certain embodiments, the system further comprises a reporting module configured to receive at least one signal from the control module, and responsively generate a user-perceptible alarm signal.

In certain embodiments, the system further comprises a reporting module configured to (i) receive at least one signal from the control module, (ii) store information indicative of or derived from the at least one signal, and (ii) generate one or more reports comprising information indicative of or derived from the at least one signal.

In certain embodiments, the at least one sensing module comprises a plurality of sensing modules.

In certain embodiments, the control module is further configured to control a HVAC apparatus associated with the conditioned indoor environment.

In another aspect, the disclosure relates to a method for mitigating airborne contamination in a conditioned indoor environment. The method comprises: detecting presence and/or concentration of particles and/or aerosols at a location in the conditioned indoor environment using at least one sensing module, wherein each sensing module of the at least one sensing module comprises an aerosol and/or particulate detector; and utilizing a control module employing an artificial intelligence algorithm to selectively activate, based on utilization of machine learning programmed rules and output signals from the at least one sensing module, at least one mitigation module configured to take one or more actions to reduce presence and/or concentration of particles and/or aerosols in the conditioned indoor environment.

In certain embodiments, the method further comprises automatically collecting an air sample from the conditioned indoor environment, using at least one sampling module, based on utilization of machine learning programmed rules and output signals from one or more modules of the at least one sensing module.

In certain embodiments, the method further comprises identifying one or more constituents of the air sample using at least one of a chemical analyzer or a biological analyzer associated with the at least one sampling module.

In certain embodiments, the at least one mitigation module comprises a ventilation module configured to increase exchange of air between the conditioned indoor environment and an outdoor environment, the ventilation module comprising an inlet fan and an outlet fan; and activating the ventilation module comprises using the outlet fan to exhaust at least a portion of air received from the conditioned indoor environment to an external environment, and comprises using the inlet fan to draw air from the external environment to the conditioned indoor environment.

In certain embodiments, the at least one mitigation module comprises a dry scrubber, and activating the dry scrubber comprises directing an air stream received from the conditioned indoor environment to contact one or more dry reagents configured to interact with constituents of the air stream.

In certain embodiments, the at least one mitigation module comprises a wet scrubber, and activating the wet scrubber comprises directing an air stream received from the conditioned indoor environment to contact one or more liquid reagents configured to interact with constituents of the air stream.

In certain embodiments, the at least one mitigation module comprises a disinfection module, and activating the disinfection module comprises activating at least one of an ozone generator or an ultraviolet lamp of the disinfection module.

In certain embodiments, the at least one mitigation module comprises a filtration module that includes a filter and a diverter, and activating the filtration module comprises activating the diverter to direct an air stream received from the conditioned indoor environment to pass through the filter.

In certain embodiments, the at least one mitigation module comprises a plurality of serially arranged mitigation modules of different types.

In certain embodiments, at least a portion of the at least one mitigation module is positioned in ductwork of an HVAC apparatus associated with the conditioned indoor environment.

In certain embodiments, the method further comprises receiving at least one signal from the control module, and responsively generating a user-perceptible alarm signal based on comparison of the at least one signal to at least one predetermined threshold value.

In certain embodiments, the method further comprises using a reporting module to receive at least one signal from the control module, to store information indicative of or derived from the at least one signal, and to generate one or more reports comprising information indicative of or derived from the at least one signal.

In certain embodiments, the at least one sensing module comprises a plurality of sensing modules.

In another aspect, the disclosure relates to a non-transitory computer readable medium containing program instructions for receiving signals from at least one sensing modules and for controlling operation of at least one mitigation module configured to take one or more actions to reduce presence and/or concentration of particles and/or aerosols in a conditioned indoor environment, to perform a method comprising: detecting presence and/or concentration of particles and/or aerosols at a location in the conditioned indoor environment using at least one sensing module that comprises an aerosol and/or particulate detector; and utilizing a control module employing an artificial intelligence algorithm to selectively activate, based on utilization of machine learning programmed rules and output signals from the at least one sensing module, the at least one mitigation module configured to take one or more actions to reduce presence and/or concentration of particles and/or aerosols in the conditioned indoor environment.

In a further aspect, any aspects, embodiments, or other features described herein may be combined for additional advantage.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiment(s), and together with the description serve to explain principles and operation of the various embodiments

### BRIEF DESCRIPTION OF DRAWINGS

FIG. 1 is a schematic diagram showing how droplets and aerosols may be propagated from an infected host (e.g., a SARS-CoV-2 infected host) to a susceptible host in an environment.

FIG. 2 is a plot of particle concentration versus time showing particle counts sensed at three different distances (3, 6, and 9 feet, respectively) from a patient undergoing nebulizer therapy.

FIG. 3 is a schematic diagram illustrating elements of a system for mitigating airborne contamination in a conditioned indoor environment according to one embodiment.

FIG. 4 is a schematic diagram illustrating elements of a system for mitigating airborne contamination in a conditioned indoor environment according to one embodiment.

FIG. 5 is a schematic diagram of components of a system for mitigating airborne contamination in a conditioned indoor environment, including components used for generating and updating an artificial intelligence algorithm employed by a control module.

FIG. 6 is a diagram showing placement of sensing modules having particle counters at different distances (i.e., 3, 6, and 13 feet, respectively) from a patient undergoing nebulizer therapy.

FIG. 7 illustrates a first graphical user interface (GUI) of a computing device (e.g., tablet computer) that may serve as a reporting module, including a memory for storing data and a display that provides recorded values (e.g., plotted with respect to time) and instantaneous values for outputs of particle/aerosol sensors of three different recording modules.

FIG. 8 illustrates a second GUI for the computing device referenced in FIG. 7 showing aerosol/particulate thresholds that, if reached, will trigger operation of fans of a ventilation module of a system for mitigating airborne contamination according to one embodiment.

FIG. 9 illustrates a third GUI for the computing device referenced in FIGS. 7-8 with the triggering of visual signals (optionally supplemented with audible signals) upon sensing by one or more sensing modules of levels of aerosols/particulates higher than one or more predetermined baseline values.

FIG. 10 is a plot of particle count per cubic feet versus time sensed by a sensing module with three aerosol/particulate sensors each including discrete capability for sensing 0.2+μm (e.g., 0.2 μm-2.0 μm size range) and 2+μm (e.g., 2.0 μm to 10.0 μm size range) particle/aerosol levels.

FIG. 11 provides a plot of three comfort parameters (temperature (F)), temperature (C), and relative humidity) sensed by one or more sensing modules for the indoor environment in a timeframe overlapping the timeframe plotted in FIG. 10.

FIG. 12 is a plot of carbon dioxide concentration sensed by one or more sensing modules for the indoor environment in a timeframe overlapping the timeframe plotted in FIG. 10.

FIG. 13 illustrates a fourth GUI 210 including a plot 212 of carbon dioxide concentration in a conditioned indoor environment with comfort parameter values (temperature, humidity, barometric pressure, and carbon dioxide concentration) obtained from a sensing module for a test performed in an office environment.

FIGS. 14A-14C represent portions of a graphical user interface, including plots of particle/aerosol concentration of two different size thresholds (0.5+μm particles and 2.5+μm particles) obtained with first through third sensing modules, respectively, during the test represented in FIG. 13.

FIG. 14D represents an additional portion of the graphical user interface supplementing the portions shown in FIGS. 14A-14C.

FIG. 15A shows a first sensing module arranged in a hallway proximate to a door of a bathroom as an example of one location for sensing module placement.

FIG. 15B shows a second sensing module mounted to a partition near shoulder level proximate to a urinal in the bathroom as another example of a location for sensing module placement.

FIG. 15C shows a third sensing module mounted to a partition near waist level proximate to a toilet in the bathroom as another example of a location for sensing module placement.

FIGS. 16A-16C represent portions of a graphical user interface, including plots of particle/aerosol concentration of two different size thresholds (0.5+ μm particles and 2.5+ μm particles) obtained with the first through third sensing modules, respectively, of FIGS. 15A-15C.

FIG. 16D represents an additional portion of the graphical user interface supplementing the portions shown in FIGS. 16A-16C.

FIG. 17A is a plot of aerosol/particulate concentration versus time obtained by a sensing module of a system as disclosed herein.

FIG. 17B shows the plot of FIG. 17A, with identification of sampling periods triggered by sensing (by one or more sensing modules) of aerosol/particulate concentration values above a baseline value.

FIG. 18 is a schematic diagram showing steps in the use of a biological sensor to provide speciation of aerosols/particulates.

FIG. 19A is a schematic illustrating components of a first biological sensor that may be incorporated in a sampling module as disclosed herein.

FIG. 19B is a schematic illustrating components of a second biological sensor that may be incorporated in a sampling module as disclosed herein.

FIG. 20 provides four representative frames from videos of mixed bacteria (E. coli) and 0.5 μm polystyrene particles in water from optical sensors shown in FIGS. 18, 19A, and 19B.

FIG. 21 is a plot of y position versus x position for bacteria and particles in the video represented in FIG. 20, showing trajectories of three bacteria and three particles.

FIGS. 22A-22C provide plots of trajectories (y position versus x position) for the three bacteria represented in FIGS. 20-21, respectively.

FIGS. 23A-23C provide plots of intensity (au) versus time for the three bacteria represented in FIGS. 20-22, respectively.

FIGS. 24A-24C provide plots of trajectories (y position versus x position) for the three bacteria represented in FIGS. 20-21.

FIGS. 25A-25C provides plots of intensity (au) versus time for the three particles represented in FIGS. 20, 21, and 24A-24C.

FIG. 26 shows components of a chemical sensor that may be incorporated in a sampling module as disclosed herein to detect metabolites of microbes.

FIG. 27 illustrates a gelatin filter impactor that may be associated with a sampling module, and useful for collection of aerosols/particles followed by transportation to a remote detector for speciation of any collected aerosols/particles.

FIG. 28 is a schematic diagram of a generalized representation of a computer system that can be utilized as, or included in a component of, a control module as disclosed herein.

FIG. 29 is a schematic diagram showing components of a system for detecting and mitigating airborne contamination with continuous monitoring and periodic sampling, with identification of steps for performing an associated method.

FIGS. 30A-30C are plots of particle concentration versus time illustrating performance of a method (for detecting and mitigating airborne contamination with continuous monitoring and periodic sampling) including steps identified in FIG. 29.

FIG. 31A illustrates a first collector and sensor assembly useable with the system of FIG. 29, including serially arranged first and second impactors, a filter, and a fan, wherein the impactors may serve as pathogen sensors.

FIG. 31B is a cross-sectional view of a portion of a collector and sensor assembly similar to that shown in FIG. 31A, showing serially arranged first and second impactors and a filter.

FIG. 32 schematically illustrates a sensor assembly for detecting SARS-CoV-2 virus particles, including a negative control area, a positive control area, and a testing area.

FIG. 33 illustrates a SARS-CoV-2 sensor image obtained with a CMOS detector, with increasing dilutions of a 525 nm quantum dot labeled protein in ionic liquid.

FIG. 34 is a calibration curve showing average intensity per exposure in seconds versus quantum dot surface area concentration (picomoles/cm²) in ionic liquid.

FIG. 35 schematically illustrates components of a wireless system for communicating outputs of multiple collector and sensor assemblies to one or more computer servers (e.g., cloud servers), wherein uploaded measurement data may be used to generate an airborne pathogen (e.g., SARS-CoV-2) map.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

As introduced previously, a system and a method for mitigating airborne contamination in a conditioned indoor environment are provided herein. Multiple sensing modules are configured to detect presence and/or concentration of particles and/or aerosols at different locations. A control module employing an artificial intelligence algorithm configured to selectively activate at least one mitigation module based on utilization of machine learning programmed rules and output signals from one or more sensing modules. The at least one mitigation module is configured to take one or more actions to reduce presence and/or concentration of particles and/or aerosols in the conditioned indoor environment. The use of an artificial intelligence algorithm employing machine learning enables mitigating actions to be taken at particularly relevant times to reduce the proliferation of microbes in an interior environment, preferably in a manner to avoid undue expenditure of energy. An interior environment may be controlled based on metrics relevant to the spread of diseases by aerosols and particulates. Mitigation measures may be utilized only when and where necessary, so that mitigation measures that consume energy are not operated on a continuous basis.

Systems and methods disclosed herein may be utilized in various structures and contexts, such as classrooms, hospitals and health-related offices and clinics, public places, government buildings, industrial buildings, airports, transportation, facilities in rural areas, retail stores, corporate facilities, and the like. In certain embodiments, wireless communication may be used between sensing modules, mitigation modules, and a control module.

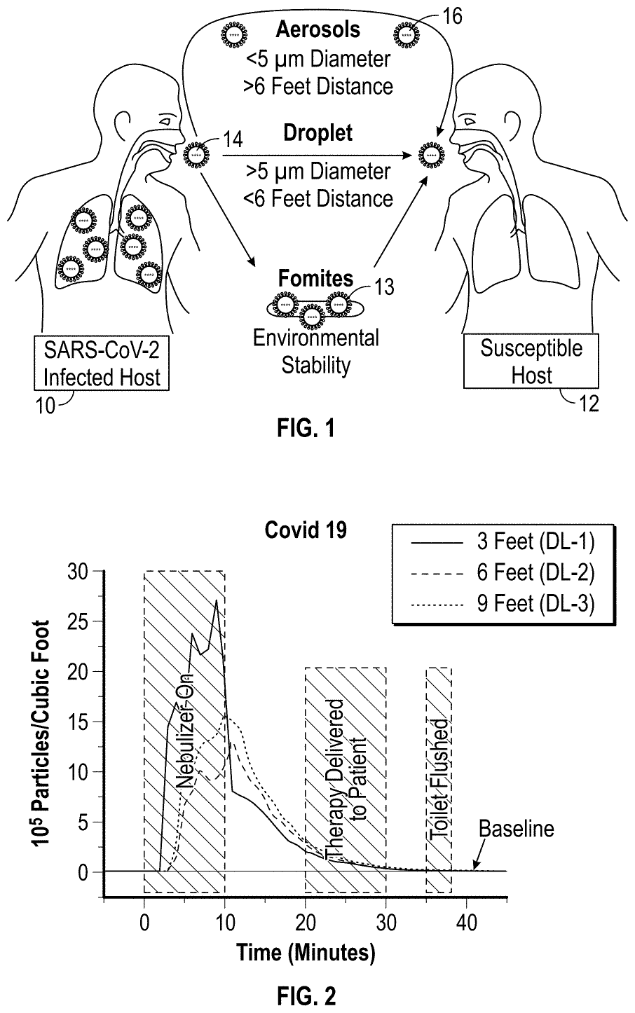

FIG. 1 is an illustrative diagram showing how droplets and aerosols may be propagated from an infected host 10 (e.g., a SARS-CoV-2 infected host) to a susceptible host 12 in an environment, and how environmentally stable fomites 23 derived from droplets 14 and/or aerosols 16 may accumulate within surfaces of the environment. FIG. 1 is adapted from the following source: T. Galbadage, B. M Peterson, R. S. Gunasekera, Does COVID-19 spread thorugh droplets alone? Front. Public Health, 24 Apr. 2020, available online at URL:<https://doi.org/10.3389/fpubh.2020.00163>.

FIG. 2 is a plot of particle concentration versus time showing particle counts sensed at three different distances (3, 6, and 9 feet, respectively) from a patient undergoing nebulizer therapy. Nebulization treatments are targeted to provide a mean of treatment for a respiratory disease. This treatment causes a high risk of spreading pathogens due to the nature of the therapy. A problem is the aerosol portions that do not reach the alveolar area, remain in the dead volume of the respiratory system (including nose and mouth) in contact with infectious areas, and are exhaled into the environment as contaminated aerosols. The “nebulizer on” portion of FIG. 2 shows particle count concentration during nebulization treatment of a patient under oxygen therapy using a high flow nasal cannula with 21% Oxygen at 30 L/min and nebulization with 3 ml of medication in a saline physiological solution. Comparative particle count profiles are illustrated for a COVID-19 infected patient at positions of 3 feet, 6 feet, and 9 feet from the subject, with the subject not wearing any mitigation mask. The spread of potentially contaminated aerosol is evident, and unpredictable. For example, aerosol concentration at a distance of 9 feet is larger than at a distance of 6 feet.

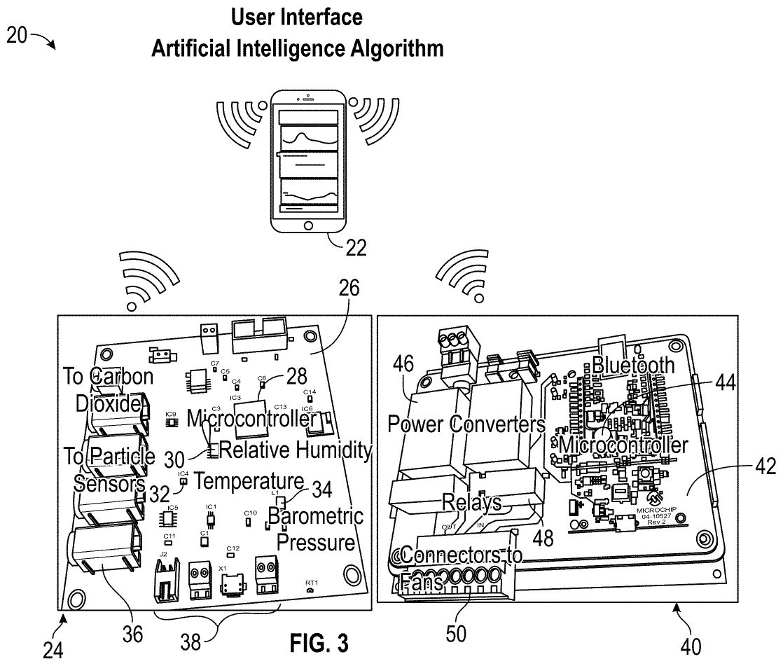

FIG. 3 is a schematic diagram illustrating elements of a system 20 for mitigating airborne contamination in a conditioned indoor environment according to one embodiment. A sensing module 24 is illustrated at lower left, with the sensing module 24 including a circuit board 26 having mounted thereon a microcontroller (or CPU) 28, and with the circuit board 26 having various sensors (e.g., relative humidity sensor 30, temperature sensor 32, barometric pressure sensor 34, etc.) mounted thereon, and additional sensors (e.g., carbon dioxide and particle sensors) coupled to the circuit board 24 via input/output ports 24. Various types of sensors that may be used include, but are not limited to, one or more particle/aerosol sensors (optionally configured to detect particles/aerosols of different sizes, such as particles/aerosols 0.2 μm or larger (e.g., 0.2 μm to 2.0 μm size range), and particles/aerosols 2.0 μm or larger (e.g., 2.0 μm to 10.0 μm size), temperature sensor, barometric pressure sensor, humidity sensor, carbon dioxide sensor, and any other sensors that may be used to detect occupancy of the environment or conditions indicative of indoor air quality and/or comfort of humans in a conditioned space where the sensing module is positioned. At least a portion of a mitigation module 40 is illustrated at lower right, with the illustrated mitigation module 40 comprising a ventilation module that is configured to increase exchange of air between the conditioned indoor environment and an outdoor environment, wherein the ventilation module 40 is configured to control (and optionally comprises) an inlet fan and an outlet fan. The ventilation module 40 may be used to increase a rate of air exchange between an indoor conditioned environment and an external environment in order to decrease a particle/aerosol level in the conditioned environment to a baseline level, responsive to detection by the sensing module 24 of particle/aerosol levels above a normal baseline level. The ventilation module 40 (as an example of a mitigation module) may include a circuit board 42, a microcontroller (or CPU) 44, power converters 46, relays 48, and electrical connectors 50 for fans (e.g., an inlet fan and an outlet fan), compressors, dampers, diverters, and/or other HVAC components. The sensing module 24 and the ventilation module 40 are arranged to communicate, either wirelessly (e.g., via Bluetooth) or by wired means, with a control module 22 that includes a microprocessor (or CPU) and memory (not shown), with the control module 22 employing an artificial intelligence (AI) algorithm configured to selectively activate the at least one mitigation (i.e., ventilation) module 40. The control module 22 and AI algorithm may utilize machine learning programmed rules as well as input signals from one or more sensing modules 24 to control operation of the mitigation module 40.

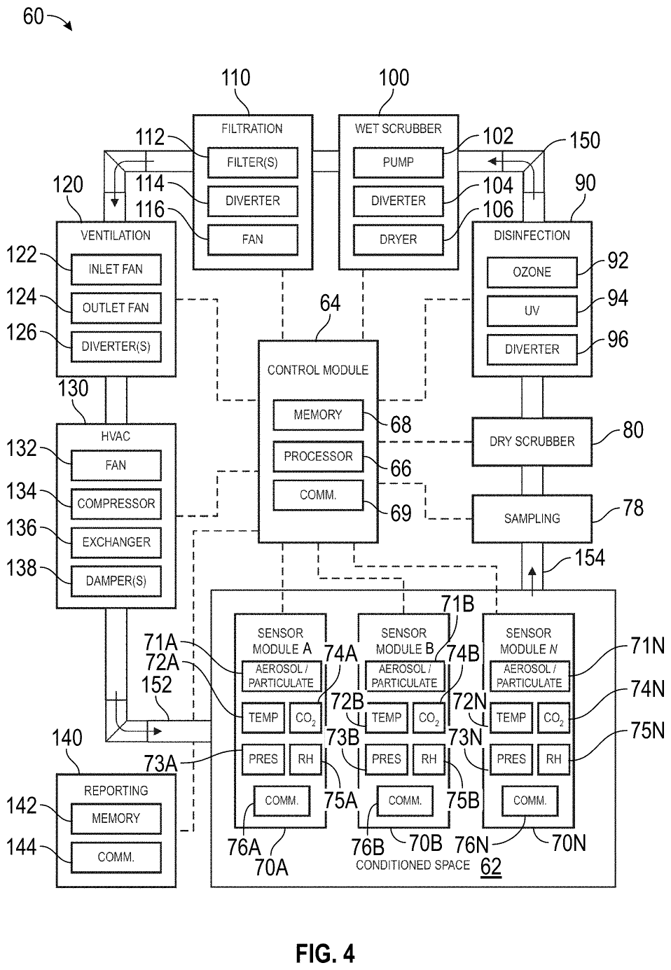

FIG. 4 is a schematic diagram illustrating elements of a system 60 for mitigating airborne contamination in a conditioned indoor environment or space 62 according to one embodiment. The system 60 includes a control module 64 having a processor 66 (e.g., a microprocessor such as a CPU), a memory 68, and a communication element 69 (e.g., Bluetooth or similar) that is operatively coupled with multiple sensing modules 70A-70N (i.e., sensing modules A to N, where N represents any suitable number), multiple mitigation modules 80, 90, 100, 110, 120, a reporting module 140, a sampling module 78, and a HVAC apparatus 130. Each sensing module 70A-70N is arranged in an indoor conditioned space 62, and may include multiple sensors 71A-76A, 71B-76B, 71N-76N. Various sensors that may be employed in each sensing module 70A-70N may include one or more particle/aerosol sensors 71A-71N (optionally including multiple particle/aerosol sensors), temperature sensors 72A-72N, barometric pressure sensors 73A-73N, humidity sensors 75A-75N, carbon dioxide sensors 74A-74N, and any other sensors that may be used to detect conditions indicative of indoor air quality and/or comfort of humans in the conditioned indoor space 62. The sensing modules 70A-70N may be arranged at different locations in the conditioned space 62. Alternatively, a single sensing module 70A may be positioned in the conditioned space, and may communicate with multiple aerosol/particle sensors that can be located at different locations in the conditioned space 62. The conditioned space 62 includes a duct loop 150 having at least one air supply duct 152 and at least one return air duct 154 that are coupled with a HVAC apparatus 130, wherein the HVAC apparatus 130 includes a fan 132, a compressor 134, a heat exchanger 136, and one or more dampers 138. At least portions of the sampling module 78 and the various mitigation modules 80, 90, 100, 110, 120 may be arranged in or proximate to the duct loop 150. The sampling module 78 may be arranged to automatically collect an air sample from an air stream received from the conditioned indoor environment 62 (e.g., via return air duct 154) based on a control signal received from the control module 64, with such control signal utilizing of machine learning programmed rules and output signals from one or more of the plurality of sensing modules 70A-70N. The sampling module 78 may be used for automatically gathering samples at critical moments of higher aerosol/particulate concentration, and may provide speciation of aerosols/particulates. In certain embodiments, the sampling module 78 may include a chemical analyzer and/or a biological analyzer (e.g., including but not limited to a specific binding assay device) configured to identify one or more constituents of a collected air sample. In certain embodiments, samples gathered by the sampling module 78 may be analyzed at an offsite facility (not shown).

The various mitigation modules 80, 90, 100, 110, 120 shown in FIG. 4 include a dry scrubber module 80, a disinfection module 90, a wet scrubber module, a filtration module, and a ventilation module. In certain embodiments, a mitigation module may include a diverter, which may include one or more dampers or other air redirecting devices that serve to direct some or all of an air stream from a primary duct loop to a secondary duct section associated with the mitigation module. Any one or more of the mitigation modules may be provided, and controlled by the control module utilizing machine learning programmed rules as well as input signals from one or more of the sensing modules. For example, if a condition indicative of high aerosol or particulate concentration in the conditioned space is identified, the control module may activate one or more of the mitigation modules in order to reduce a concentration of aerosols or particulates in the conditioned space. In certain embodiments, activating the dry scrubber comprises directing an air stream received from the conditioned indoor environment to contact one or more dry reagents configured to interact with constituents of the air stream. In certain embodiments, activating the disinfection module comprises activating at least one of an ozone generator or an ultraviolet lamp of the disinfection module, possibly in conjunction with operating a diverter of the disinfection module. In certain embodiments, activating the wet scrubber module comprises activating a diverter, a liquid reagent pump, and a dryer, to cause at least a portion of an air stream to interact with a liquid reagent followed by drying of a wetted air stream to reduce concentration of aerosols or particulates. In certain embodiments, activating the filtration module comprises activating a diverter to direct at least a portion of an air stream from a primary loop to a secondary loop containing a high efficiency filter (e.g., a HEPA filter or bacterial/viral filter), optionally in conjunction with activating a filtration fan to force diverted air through the high efficiency filter. In certain embodiments, activating the ventilation module comprises operating an outlet fan to exhaust at least a portion of air received from the conditioned indoor environment to an external environment, and comprises operating an inlet fan to draw air from the external environment to the conditioned indoor environment, optionally in conjunction with operating a diverter or other airflow control apparatus to prevent an air stream from bypassing the inlet and outlet fans or a scrubber filtering system 9 e.g., bacterial/viral filter, activated carbon filter, etc.) to ensure the entrance of clean air. The reporting module may include a memory and a communication element, optionally in conjunction with a display. In certain embodiments, the reporting module is configured to receive at least one signal from the control module, and responsively generate a user-perceptible alarm signal. In certain embodiments, the reporting module is configured to (i) receive at least one signal from the control module, (ii) store information indicative of or derived from the at least one signal, and (ii) generate one or more reports comprising information indicative of or derived from the at least one signal. The reports can be audible (e.g., alarms), visual (e.g., color coded displayed signals), and/or tactile (e.g., vibration).

In certain embodiments, an AI algorithm utilized by a control module comprises a neural network algorithm inspired by biological neurons. A deep neural network may utilize many layers of connected neurons in sequence. An exemplary neural network may include an input layer, one or more hidden layers, and an output layer.

In certain embodiments, an AI algorithm utilized by a control module employs machine learning, which may include supervised, unsupervised, semi-supervised, and/or reinforcement learning. An AI algorithm built with machine learning may be generated by providing prepared training date to an AI algorithm. Such a process may include gathering raw data, preparing training data, training and optimizing an AI model, integrating an AI model, testing/evaluating an AI model, and placing an AI algorithm (obtained from the AI model) in operational use. In certain embodiments, data obtained through operational use of an AI algorithm may be used to prepare additional training data for further refinement and/or updating of the AI algorithm.

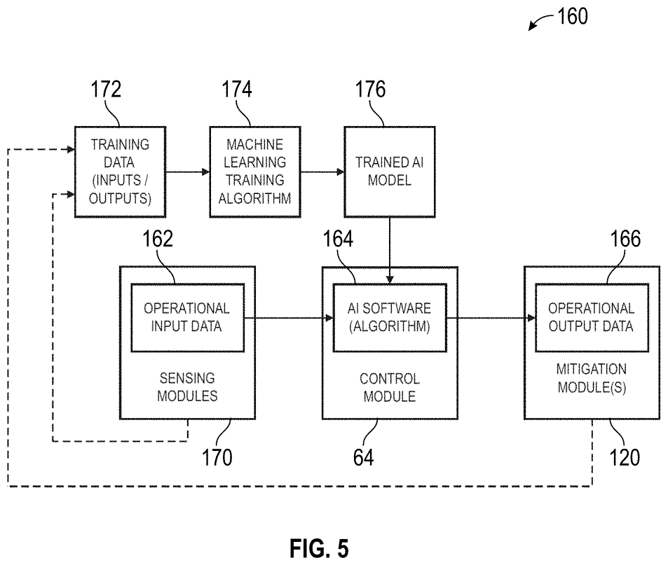

FIG. 5 is a schematic diagram of components of a system 160 for mitigating airborne contamination in a conditioned indoor environment, including components used for generating and updating an artificial intelligence algorithm employed by a control module. The system 160 includes sensing modules 170, a control module 64, and one or more mitigation modules 120. The sensing modules 170 provide operational input data 162 to the control module 64, which operates an AI algorithm 164 that may be implemented in AI software. Through operation of the AI algorithm 164, the control module 64 provides operational output data 166 (e.g., control signals) to one or more mitigation modules 120. Although not shown in FIG. 5, the control module 64 may also provide signals to a reporting module, a sampling module, and/or a HVAC apparatus (e.g., as depicted in FIG. 4). To generate an AI algorithm 164, training data 172 (e.g., input and output data) may be supplied to a machine learning training algorithm 174 to produce a trained AI model 176. After sufficient machine learning training is complete (and any desired testing and validation is completed), the trained AI model 176 may be placed into operational use as the AI algorithm 164 used by the control module 64. In certain embodiments, data obtained during operational use of the AI algorithm 164 (e.g., operational input data 162 generated by the sensing modules 170, and operational output data 166 provided to the mitigation modules 120) may be used to prepare additional training data for further refinement and/or updating of the AI algorithm 164 employed by the control module 64.

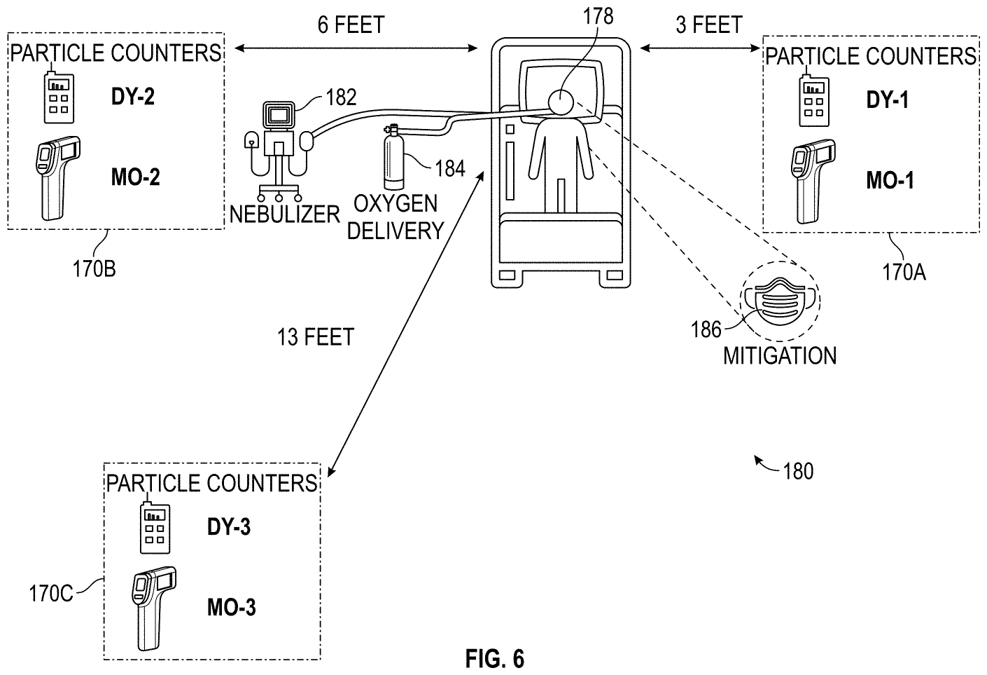

FIG. 6 is a diagram showing placement of sensing modules 170A-170C having particle sensors (e.g., counters) at different distances (i.e., 3, 6, and 13 feet, respectively) from a patient 178 undergoing nebulizer therapy via a nebulizer 182 incorporating oxygen delivery 184, wherein the patient 178 may have associated therewith exposure mitigation equipment 186 such as a mask, filter, and/or limited air exchange apparatus. Sensing modules 170A-170C are desirably placed at different locations in a conditioned environment 180, since aerosol and/or particulate concentration may vary considerably within the conditioned environment 180 due to flows of air generated by a HVAC system.

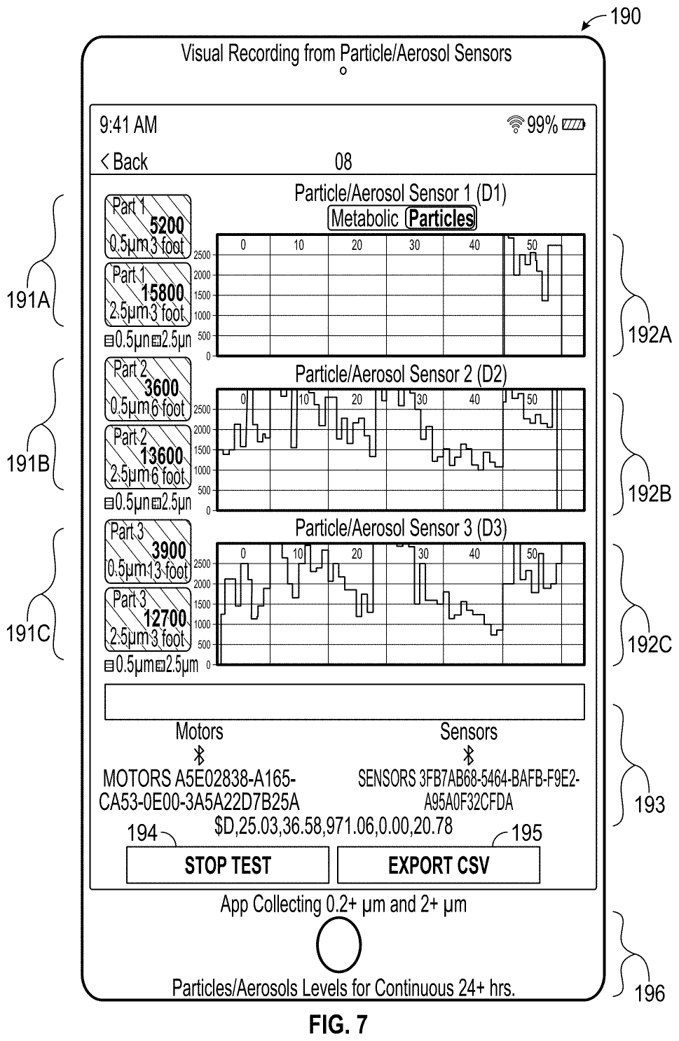

FIG. 7 illustrates a first graphical user interface (GUI) 190 of a computing device (e.g., tablet computer) that may serve as a reporting module, with the computing device including a memory for storing data and a display that provides recorded values (e.g., plotted with respect to time) and instantaneous values for outputs of particle/aerosol sensors of three different recording modules. For example, an application may collect and display 0.2+ μm and 2+ μm particle/aerosol levels over various timeframes, such as hourly, 24 hours, weekly, monthly, etc. The first GUI 190 includes instantaneous readings 191A-191C for three particle sensors arranged at different distances (i.e., 3, 6, and 13 feet, respectively) from a patient, and further includes time-varying plots 192A-192C for outputs of these sensors. The first GUI 190 additionally includes an equipment identification window 193 (showing associated motor and sensor identifiers), a stop test button 194, an export data (export CSV) button 195, and a setting status window 196.

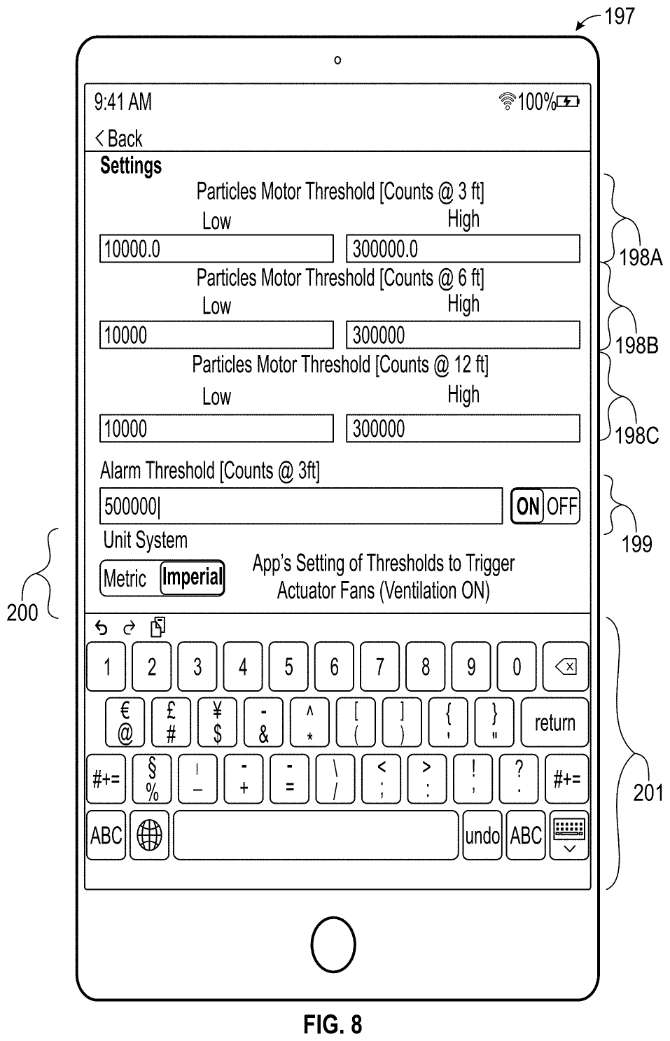

FIG. 8 illustrates a second GUI for the computing device referenced in FIG. 7, showing user-settable aerosol/particulate threshold windows 198A-198C (for particle counts obtained by sensors distanced 3 feet, 6 feet, and 12 feet from a patient or location of interest), an alarm threshold window 199, and a keyboard window 201. Any one or more of the high thresholds in windows 198A-198C, 199 may be utilized for trigger operation of fans of a ventilation module of a system for mitigating airborne contamination according to one embodiment.

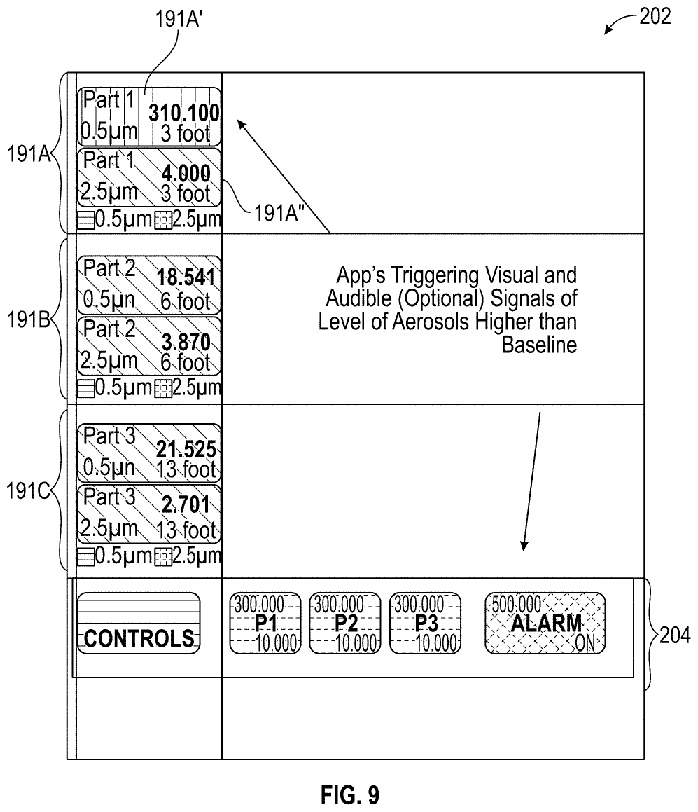

FIG. 9 illustrates a third GUI 202 for the computing device referenced in FIG. 7, showing instantaneous particle sensing windows 191A-191B that may be shaded or colored to provide visual signals (optionally supplemented with audible signals) if a particle count threshold is attained by sensing with one or more sensing modules corresponding to levels of aerosols/particulates higher than one or more predetermined baseline or threshold values. The third GUI further includes threshold status windows 204 that may be used to identify currently set high and low threshold values.

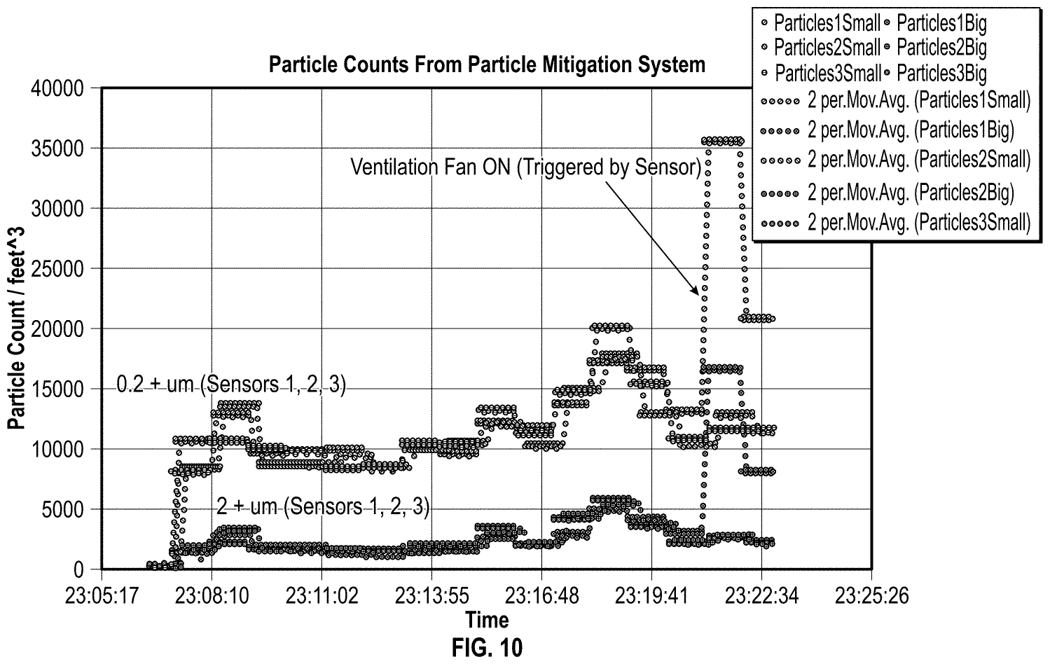

FIG. 10 is a plot of particle count per cubic feet versus time sensed by a sensing module with three aerosol/particulate sensors each including discrete capability for sensing 0.2+ μm (e.g., 0.2 μm-2.0 μm size range) and 2+ μm (e.g., 2.0 μm to 10.0 μm size range) particle/aerosol levels. As shown, spikes in detected 0.2+ μm particles and 2+ μm particles around 23:20:00 triggers operation of a ventilation fan of a ventilation module, in order to promote exchange air between a conditioned indoor environment and an outdoor environment, in order to reduce concentration of aerosols/particles in the conditioned indoor environment. When the detected concentration of aerosols/particles returns to acceptable levels, operation of the ventilation module may be discontinued as unnecessary, until another spike in aerosols/particles is detected.

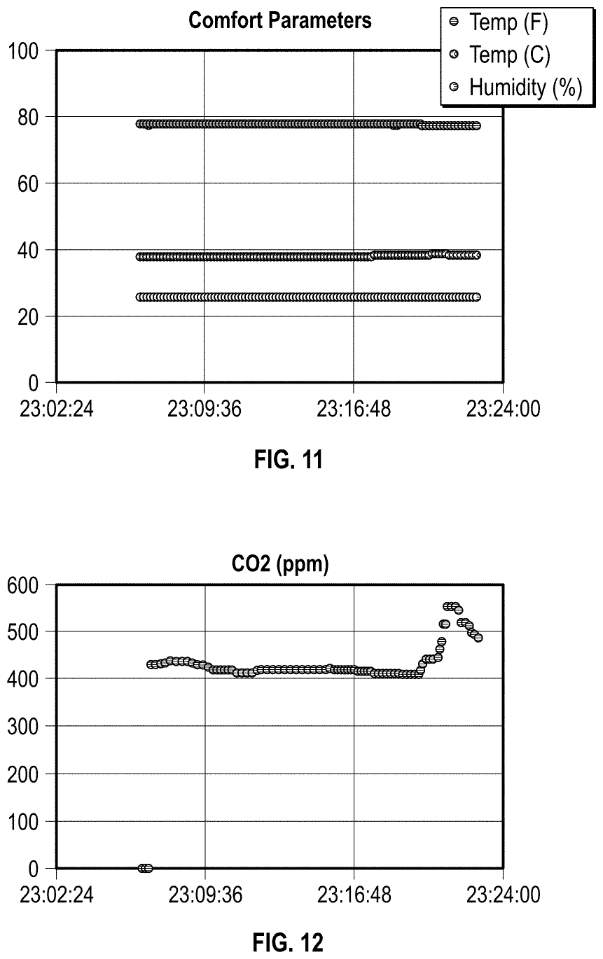

FIG. 11 provides a plot of three comfort parameters (temperature (F)), temperature (C), and relative humidity) sensed by one or more sensing modules for the indoor environment in a timeframe overlapping the timeframe plotted in FIG. 10. As shown, temperature and relative humidity values remain stable over the displayed time period.

FIG. 12 is a plot of carbon dioxide concentration sensed by one or more sensing modules for the indoor environment in a timeframe overlapping the timeframe plotted in FIG. 10. As shown, carbon dioxide concentration values remain stable over the displayed time period.

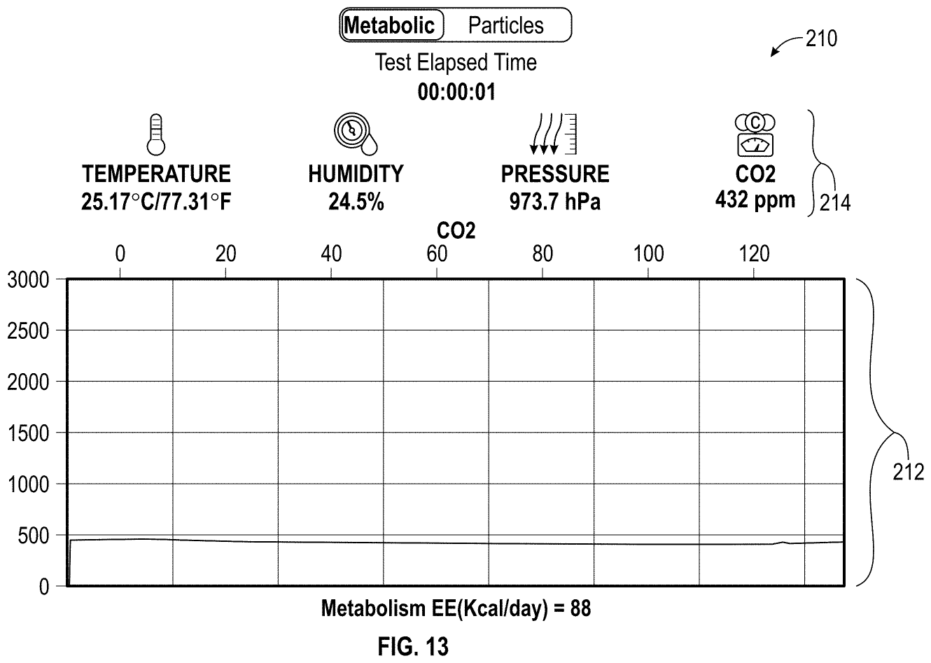

FIG. 13 illustrates a fourth GUI 210 including a plot 212 of carbon dioxide concentration in a conditioned indoor environment, with additional windows 214 providing instantaneous readings of comfort parameter values (temperature, humidity, barometric pressure, and carbon dioxide concentration) obtained from a sensing module for a test performed in an office environment. The carbon dioxide levels analysis may be associated to an AI algorithm measuring the metabolic rate (kcal/day) of a sole occupant in the environment.

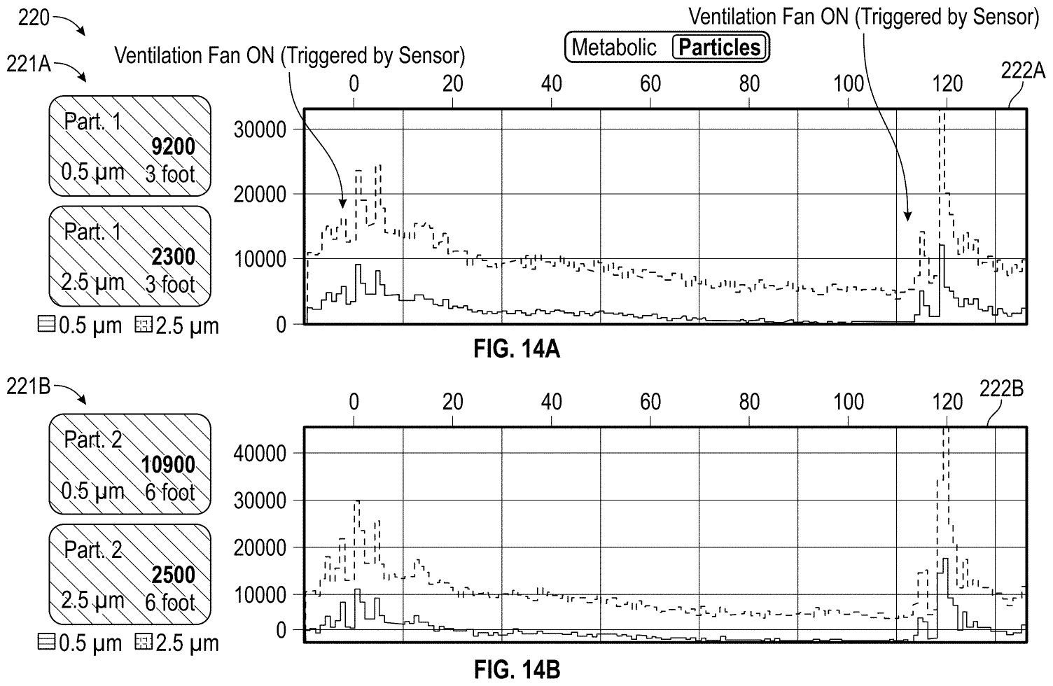

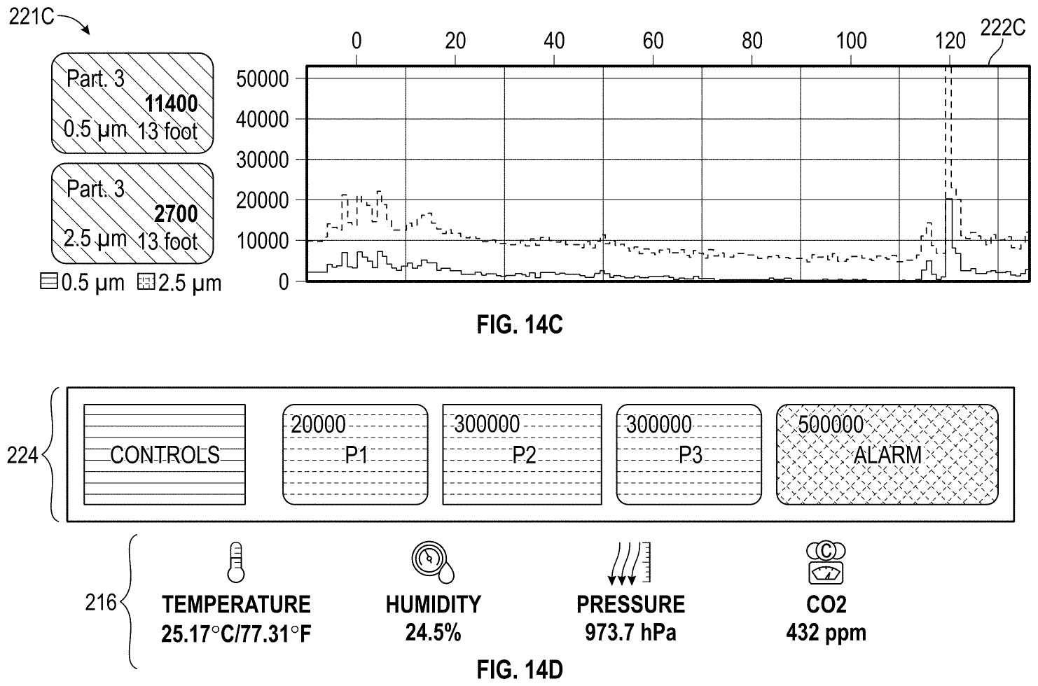

FIGS. 14A-14C represent portions of a GUI 220 useable with a computing device connected to sensing modules described herein, with each figure including instantaneous reading windows 221A-221C and time-varying plots 222A-222C of particle/aerosol concentration of two different size thresholds (0.5+ μm particles and 2.5+ μm particles) obtained with first through third sensing modules, respectively, during the test represented in FIG. 13. FIG. 14A provides values for a sensing module positioned 3 feet from a patient or location of interest, FIG. 14B provides values for a sensing module positioned 6 feet from the patient or location of interest, and FIG. 14C provides values for a sensing module positioned 13 feet from the patient or location of interest, Such figures show the triggering of a ventilation module at two time periods (proximate to time=0 and time=117 minutes) responsive to detection of elevated particulate/aerosol concentration values.

FIG. 14D represents an additional portion of the graphical user interface 220 supplementing the portions shown in FIGS. 14A-14C, including a control and threshold/alarm identification window 224, and additional windows 216 providing instantaneous readings of comfort parameter values (temperature, humidity, barometric pressure, and carbon dioxide concentration).

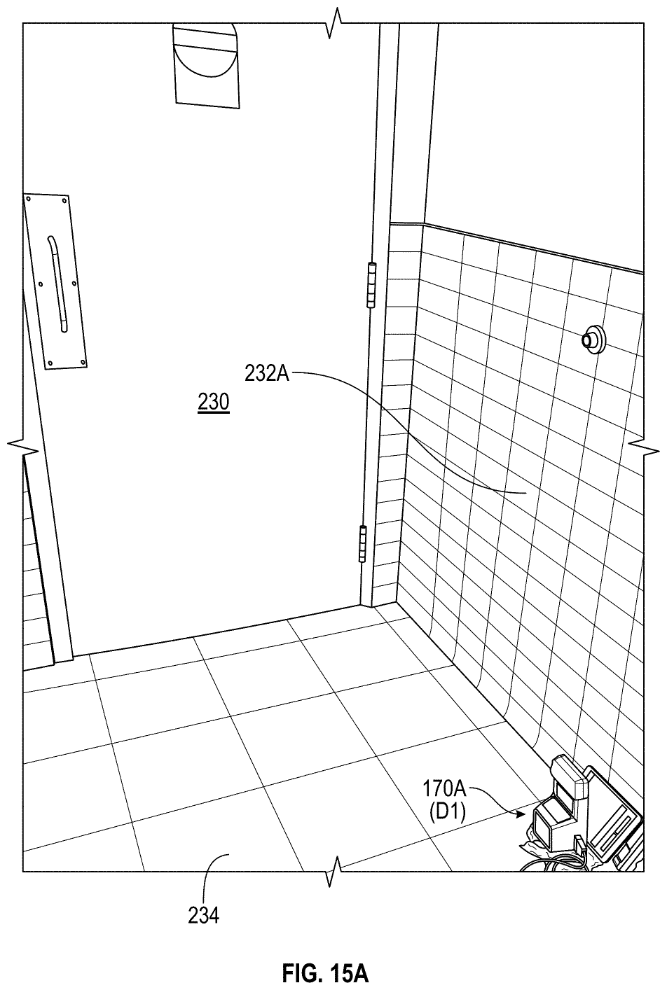

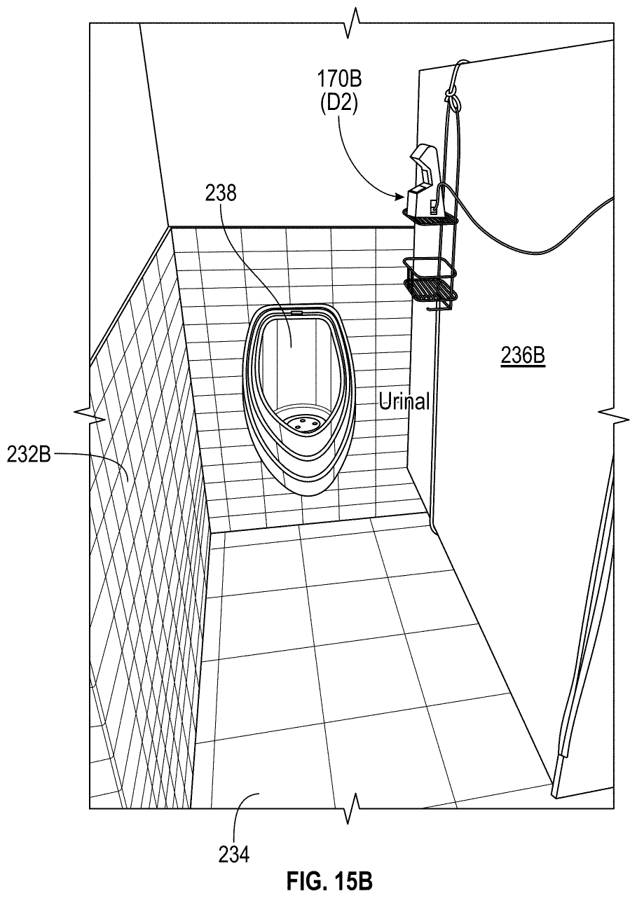

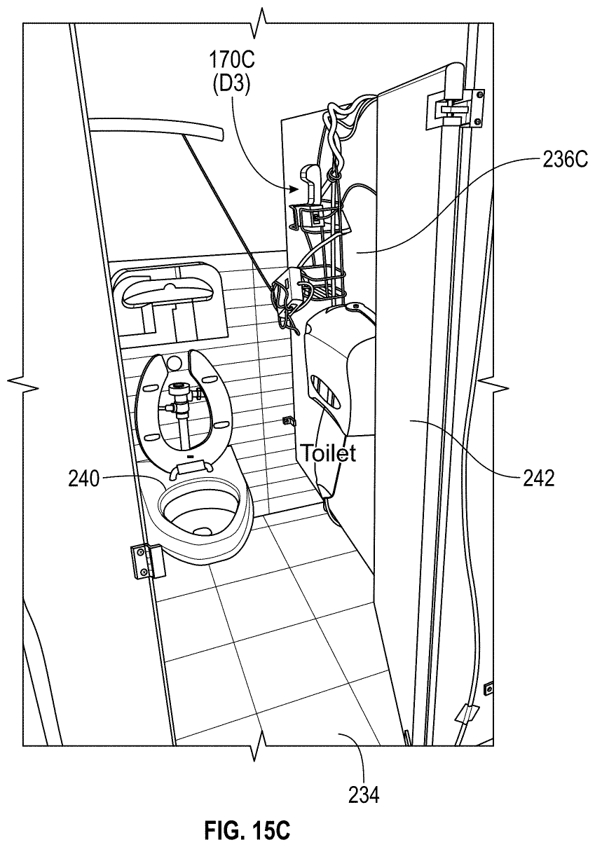

FIGS. 15A-15C illustrate placement of sensing modules at different locations in a bathroom. FIG. 15A shows a first sensing module 170A arranged in a hallway proximate to a door 230 and floor 234 (and adjacent to a wall 232A) of a bathroom as an example of one location for sensing module placement. FIG. 15B shows a second sensing module 1708 mounted to a partition 236B near shoulder level proximate to a urinal 238 (located between the partition 236B and an opposing wall 232B, and elevated above a floor 234) in the bathroom as another example of a location for sensing module placement. FIG. 15C shows a third sensing module 170C mounted to a partition 236C (elevated above a floor 234) and near waist level proximate to a toilet 240 in a stall (having a door 242) in the bathroom as another example of a location for sensing module placement. The different placement of the sensing modules 170A-70C in FIGS. 15A-15C is expected to yield different sensed values for particulate/aerosol concentration in the same room.

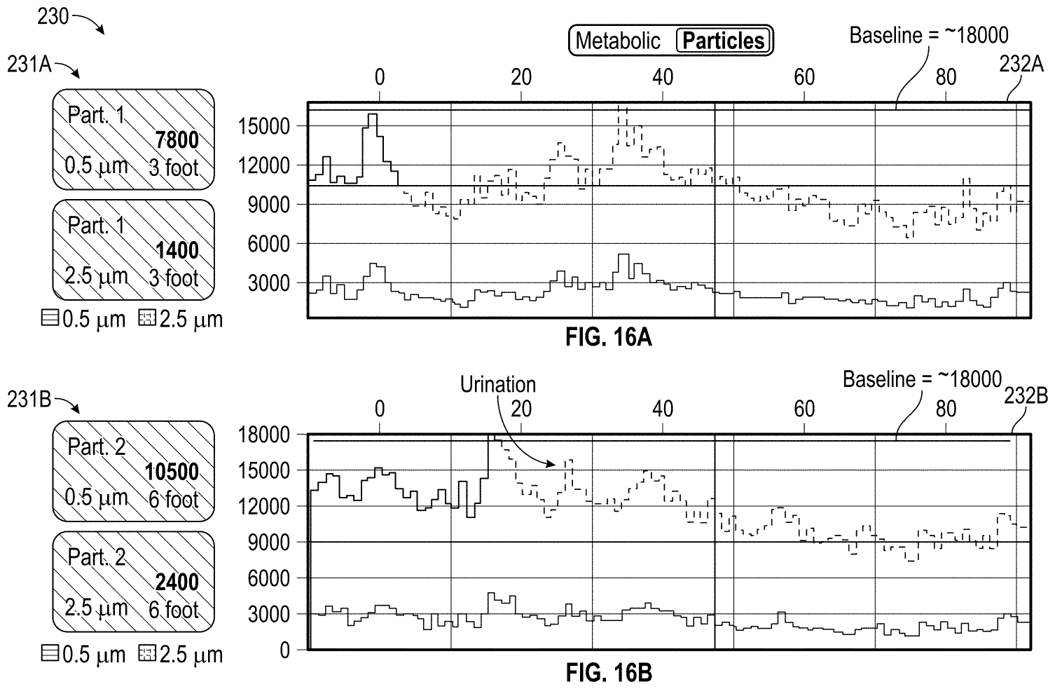

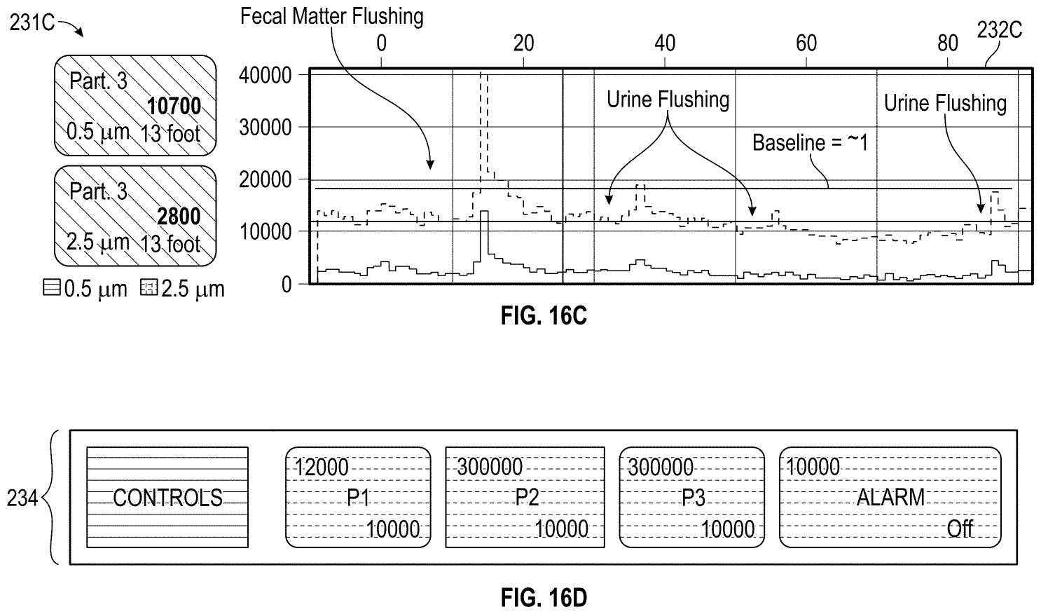

FIGS. 16A-16C represent portions of a GUI 230 useable with a computing device connected to sensing modules described herein, with each figure including instantaneous reading windows 221A-221C and time-varying plots 232A-232C of particle/aerosol concentration of two different size thresholds (0.5+ μm particles and 2.5+ μm particles) obtained with first through third sensing modules 170A-170C of FIGS. 15A-15C. FIG. 14A provides values for a sensing module positioned 3 feet from a location of interest, FIG. 14B provides values for a sensing module positioned 6 feet from a location of interest, and FIG. 14C provides values for a sensing module positioned 13 feet from a location of interest. FIGS. 14A-14C show detected spikes in aerosol/particulate concentration at different times, corresponding to events such as urination in the urinal (see FIG. 16B), fecal matter flushing in the toilet (see FIG. 16C), and urine flushing in the toilet (see FIG. 16C), wherein operation of a ventilation module is triggered at two time periods responsive to detection of elevated particulate/aerosol concentration values. The first sensing module (in FIG. 16A) shows particle/aerosol concentration values below a baseline (18,000 particles/ft³) at all times. The second sensing module (in FIG. 16B) also shows particle/aerosol concentration values below a baseline (18,000 particles/ft³) at all times, demonstrating that urination in a chemical urinal does not produce unduly high aerosol concentrations. The third sensing module (in FIG. 16C) shows spikes in particle/aerosol concentration every time the toilet is flushed, and values clearly above the baseline when flushing is associated with disposal of fecal matter. In view of the foregoing, in certain embodiments, an AI algorithm receiving data from sensing modules in a bathroom environment may be used to responsively and/or prophylactically initiate one or more mitigation modules when a fecal matter flushing event is detected or is considered to be imminent (e.g., by detection of a condition indicative of an occupant seated on a toilet, or other conditions).

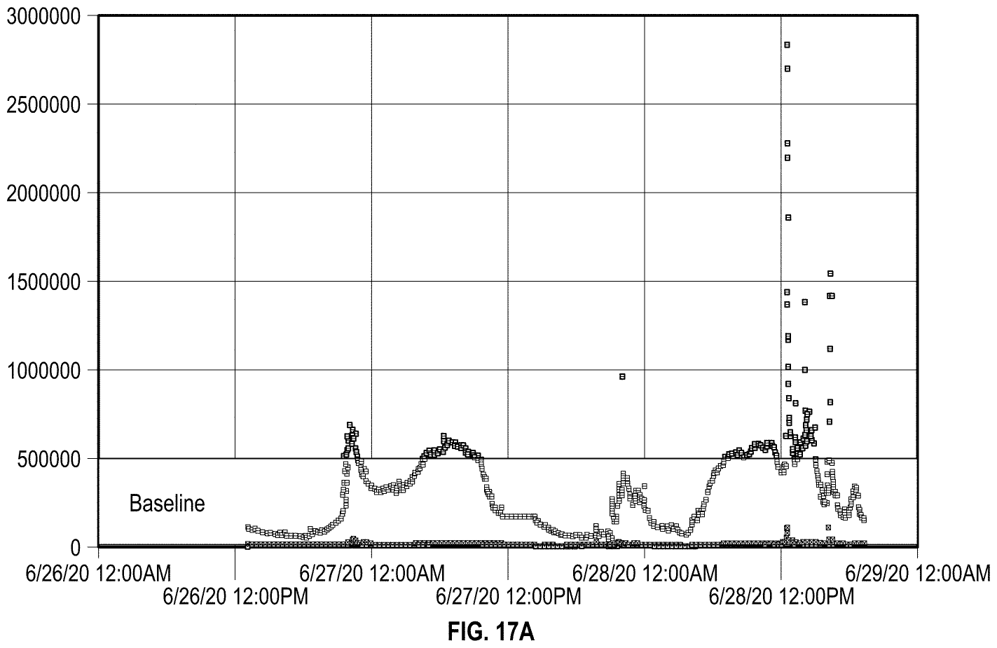

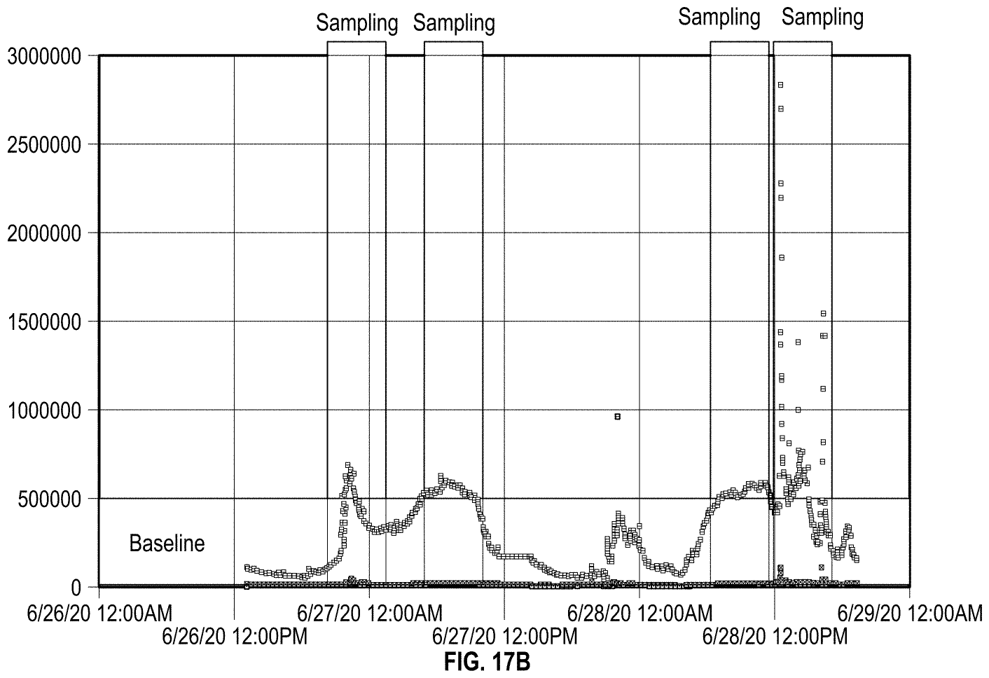

FIG. 17A is a plot of aerosol/particulate concentration versus time obtained by a sensing module of a system as disclosed herein, taken over a period of 54 hours and including a baseline value threshold region (i.e., for concentration values below 500000). As shown, three time regions exceed the baseline region, with the latter time region (on Jun. 28, 2020) exhibiting the highest overage suitable for triggering a sampling period.

FIG. 17B shows the plot of FIG. 17A, with identification of sampling periods triggered by sensing (by one or more sensing modules) of aerosol/particulate concentration values above a baseline value. Four sampling periods (windows) are shown. A sampling module may be used for gathering samples at critical moments of higher aerosol/particulate concentration, and may provide speciation of aerosols/particulates. In certain embodiments, a sampling module may include a chemical analyzer and/or a biological analyzer (e.g., including but not limited to a specific binding assay device) configured to identify one or more constituents of a collected air sample.

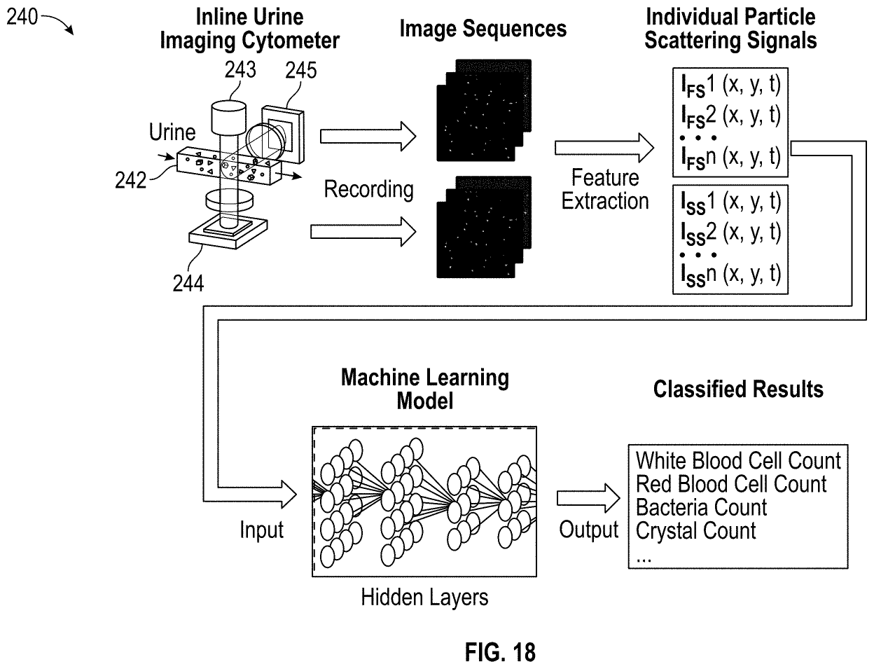

FIG. 18 is a diagram showing steps using a biological sensor 240 to provide speciation of aerosols/particulates. The illustrated biological sensor 240 comprises an inline urine imaging cytometer (i.e., fluid imaging meter) that utilizes a laser 243, a forward scattering CMOS imager 244, and a side scattering CMOS imager 254 to generate images of a fluid sample (e.g., urine) contained in a sample channel 242, within such image generation involving a first step. A second step involves recording and sequencing the images obtained from the CMOS imagers 244, 254. A third step includes extracting features from the sequenced images to generate individual particle scattering signals. A fourth step includes suppling the individual particle scattering signals as inputs to a machine learning model that employs multiple hidden layers between an input layer and an output layer. A fifth step includes classifying results obtained from the machine learning model (e.g., counts of particulate elements, and discrimination of type of particle, such as white blood cell, red blood cell, bacteria, crystal, etc.). FIG. 18 was adapted from the following source: Rafael Iriya, Wenwen Jing, Karan Syal, Manni Mo, Chao Chen, Hui Yu, Shelley E Haydel, Shaopeng Wang, Nongjian Tao, Rapid antibiotic susceptibility testing based on bacterial motion patterns with long short-term memory neural networks, IEEE Sensors Journal, vol. 20, no. 9, pp. 4940-4950, May 1, 2020. NIHMS1588088.

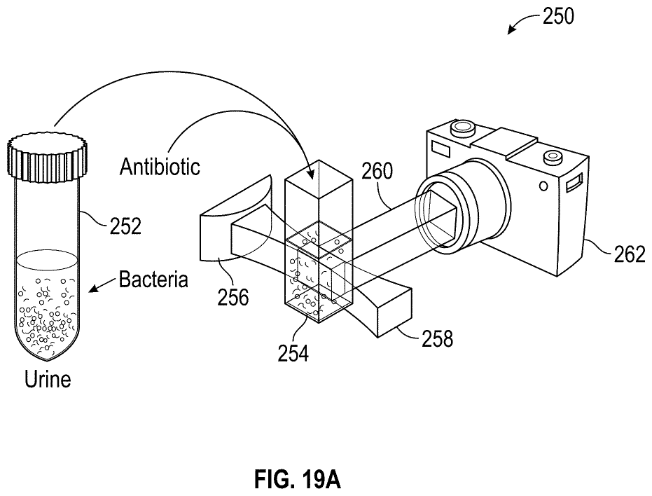

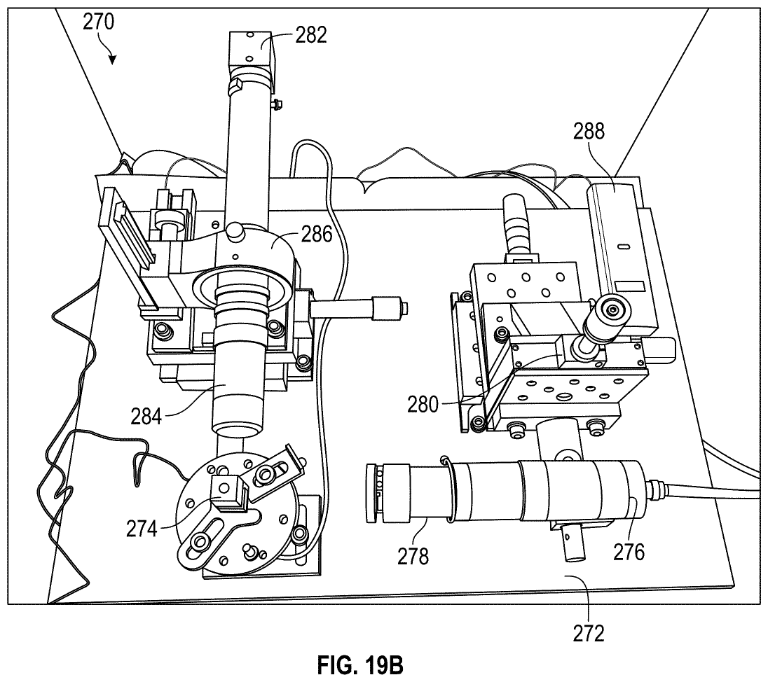

FIG. 19A shows components of a first biological sensor 250 that may be incorporated in a sampling module as disclosed herein, including a sample holder 254 (e.g., for receiving a urine sample from a via 252, plus an optionally added antibiotic) that is arranged between a light slab 256 and an optical assembly 256. Scattered light 260 produced by the illuminated sample holder 254 is received by a camera 262 to produce an output signal.

FIG. 19B shows components of a second biological sensor 270 that may be incorporated in a sampling module as disclosed herein, with the sensor 270 including a sample container 274 arranged to be illuminated by a laser 276 emitting through a cylindrical lens 278, and including a zoom lens 284 and an associated camera 282 arranged orthogonally to the cylindrical lens 278 to capture images of the sample within the sample container 274. A first translation stage 280 is associated with the cylindrical lens 278 to adjust illumination of the sample container 274, and a second translation stage 286 is associated with zoom lens 284 to facilitate imaging using the camera 282. A temperature sensor 288 is additionally provided. FIG. 19B is adapted from the following source: M Mo, Y Yang, F Zhang, W Jing, R Iriya, J Popovich, S Wang, T Grys, S. E. Haydel, N. Tao, Rapid Antimicrobial Susceptibility Testing of Patient Urine Samples using Large Volume Free-Solution Light Scattering Microscopy, Analytical chemistry, 2019, 91 (15), 10164-10171. DOI: 10.1021/acs.analchem.9b02174. PMCID: PMC7003966.

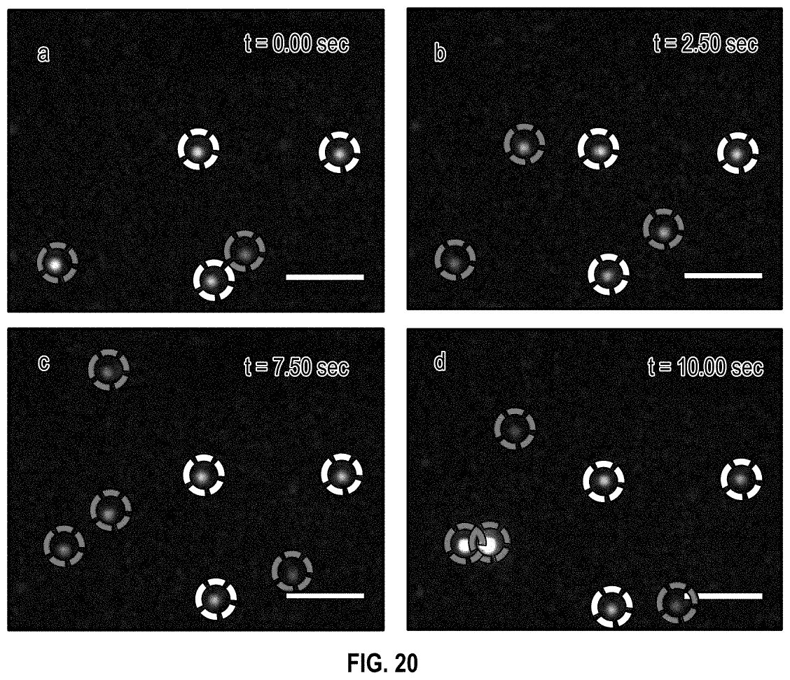

FIG. 20 provides four representative frames from videos of mixed bacteria (E. coli) and 0.5 μm polystyrene particles in water from optical sensors shown in FIGS. 18, 19A, and 19B, wherein bacteria cells are highlighted in gray dashed line circles, and particles are highlighted in white dashed line circles, with a scale bar showing a 50 μm scale. A comparison of the four frames shows that the bacteria cells exhibit greater movement (change in position) than the particles.

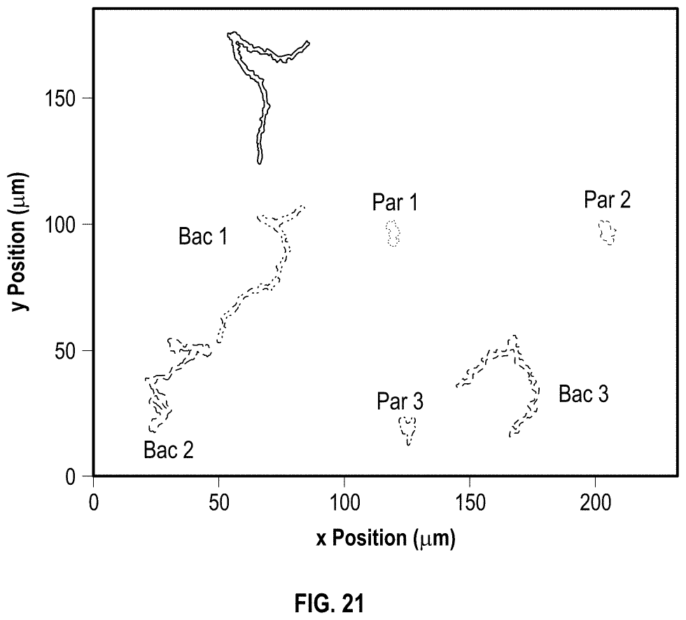

FIG. 21 is a plot of y position versus x position for bacteria and particles in the video represented in FIG. 20, showing trajectories of three bacteria (Bac1 to Bac 3) and three particles (Par1 to Par3).

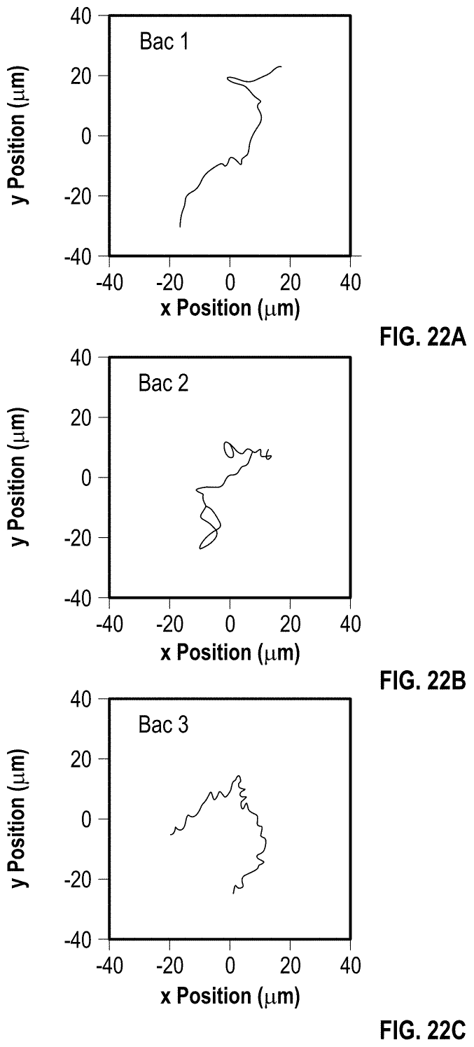

FIGS. 22A-22C provide plots of trajectories (y position versus x position) for the three bacteria (Bac1 to Bac3) represented in FIGS. 20-21, respectively.

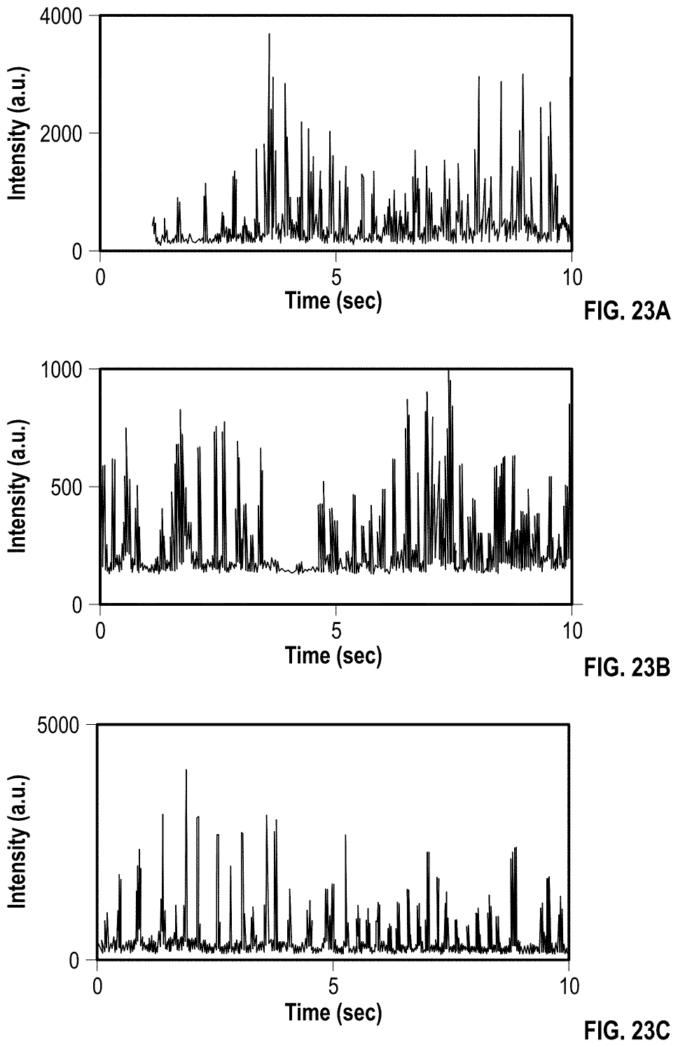

FIGS. 23A-23C provide plots of intensity (au) versus time for the three bacteria represented in FIGS. 20-22C, respectively.

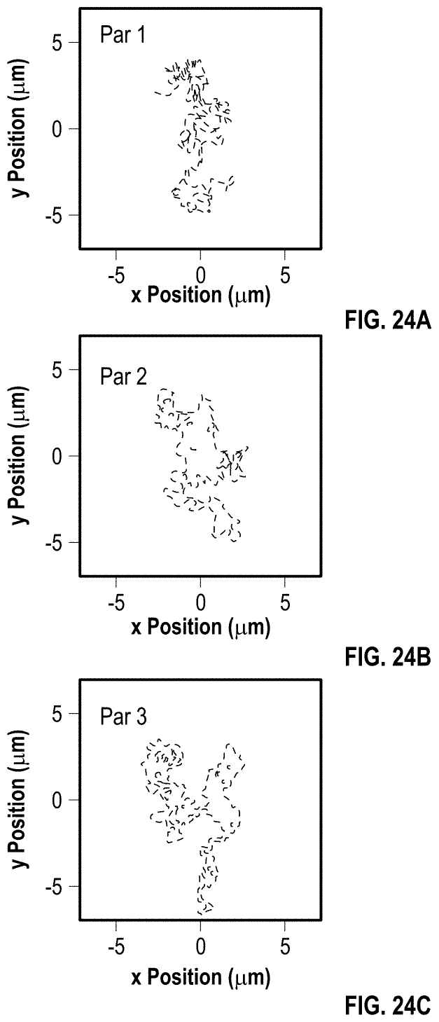

FIGS. 24A-24C provide plots of trajectories (y position versus x position) for the three particles represented in FIGS. 20-21. The particle trajectories shown in FIGS. 24A-24C have a smaller positional variation and are significantly different from the bacteria trajectories shown in FIGS. 22A-22C.

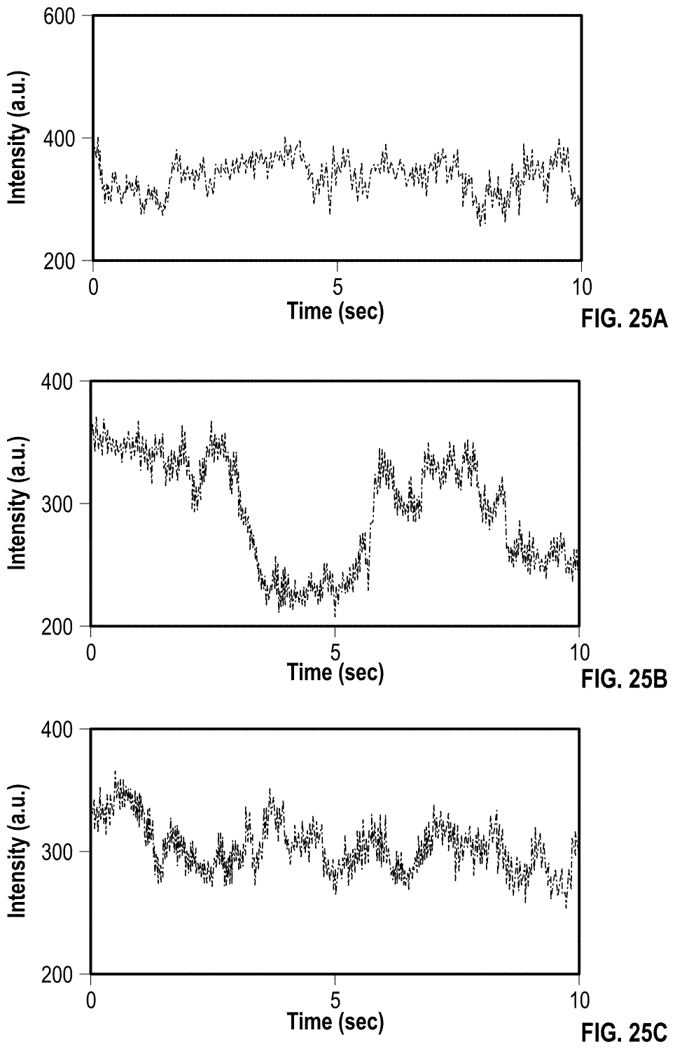

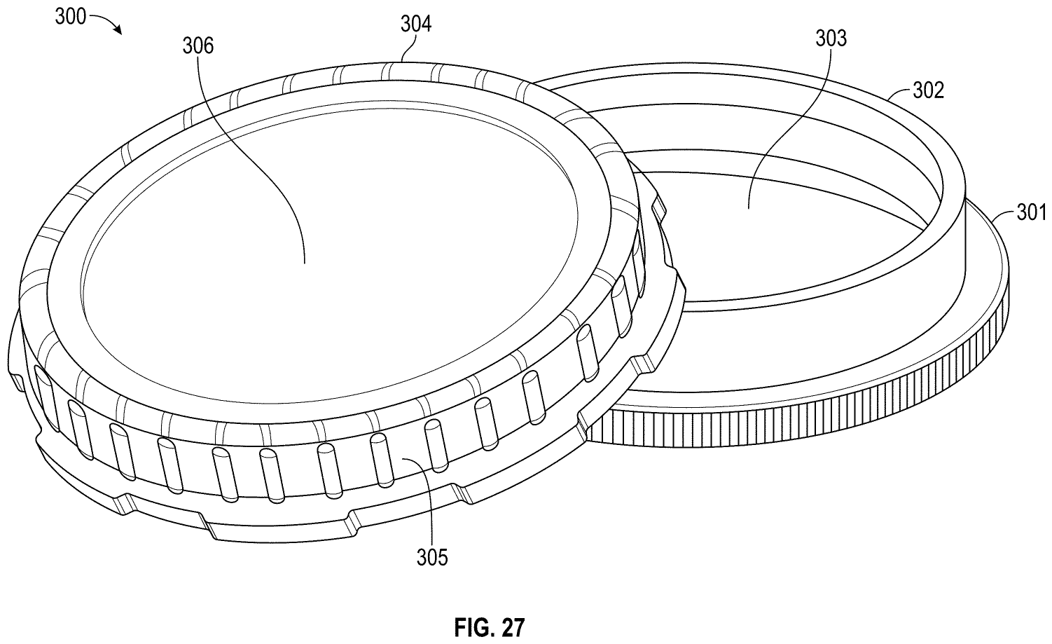

FIGS. 25A-25C provides plots of intensity (au) versus time for the three particles represented in FIGS. 20, 21, and 24A-24C. The intensity variation magnitude and patterns of FIGS. 25A-25C for particles differ significant from their counterparts shown in FIGS. 23A-23C for bacteria.

FIGS. 20-25 demonstrate the capacity of optical system such as the one shown in FIGS. 18, 19A, and 19B to discriminate bacteria from particles based on the imaging sensor signal processing.

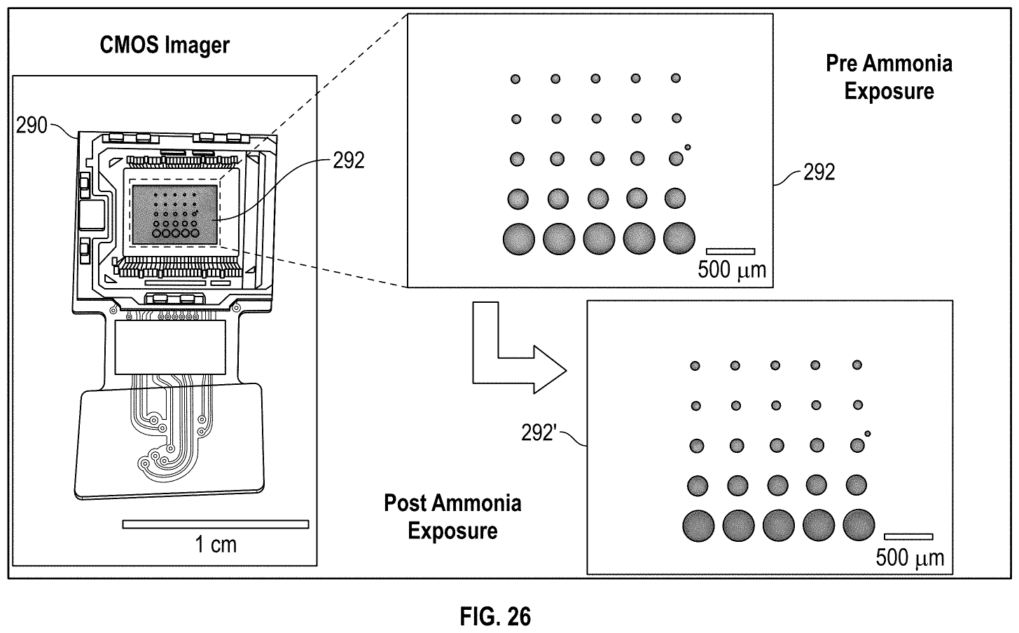

FIG. 26 shows components of a chemical sensor 290 that may be incorporated in a sampling module as disclosed herein to detect metabolites of microbes. The chemical sensor 290 may include a CMOS image chip 292 or equivalent optical system, which may be used to identify a change in state of the sensor upon exposure to one or more chemical species (e.g., ammonia as illustrated, or others such as phenol p-cresol, indole, hydrogen sulfide, nitrite, nitrate, methane, etc.). The frame at upper right in FIG. 26 shows the CMOS image chip 292 with multiple sensing areas represented in a first state (e.g., color distribution) prior to exposure to ammonia. The frame at lower right in FIG. 26 shows the CMOS image chip 292′ in a second state, with sensing areas having a different color distribution after exposure to ammonia. FIG. 26 was adapted from the following source: Kyle R. Mallires, Di Wang, Peter Wiktor and Nongjian Tao, A Microdroplet-Based Colorimetric Sensing Platform on a CMOS Imager Chip, Anal. Chem. 2020, 92, 9362-9369.

FIG. 27 illustrates a gelatin filter impactor 300 that may be associated with a sampling module, and may be used for collection of aerosols/particles followed by transportation to a remote detector for speciation of any collected aerosols/particles. The gelatin filter impactor 300 includes a body 301 having a raised wall 302 that contains a cavity 303 configured to hold gelatin or another cell culturing medium. The gelatin filter impactor 300 additionally includes a lid 304 having a wall structure 305 configured to cooperated with the body 301 and/or raised wall 302, and includes filtration media 306 spanning at least a portion of the lid 304. In use, air can be drawn through the filtration media 306 (e.g., using a vacuum pump applied to the body 301 or other means) into the gelatin filter impactor 300, to permit aerosols and/or particles to contact gelatin within the cavity 303 so that species within the aerosols and/or particles may be cultured for further analysis. In certain embodiments, the filtration media 306 may have geometric characteristics (e.g., pore shape, pore size, pore distribution, etc.) selected to promote preferential passage of species of interest into the cavity 303.

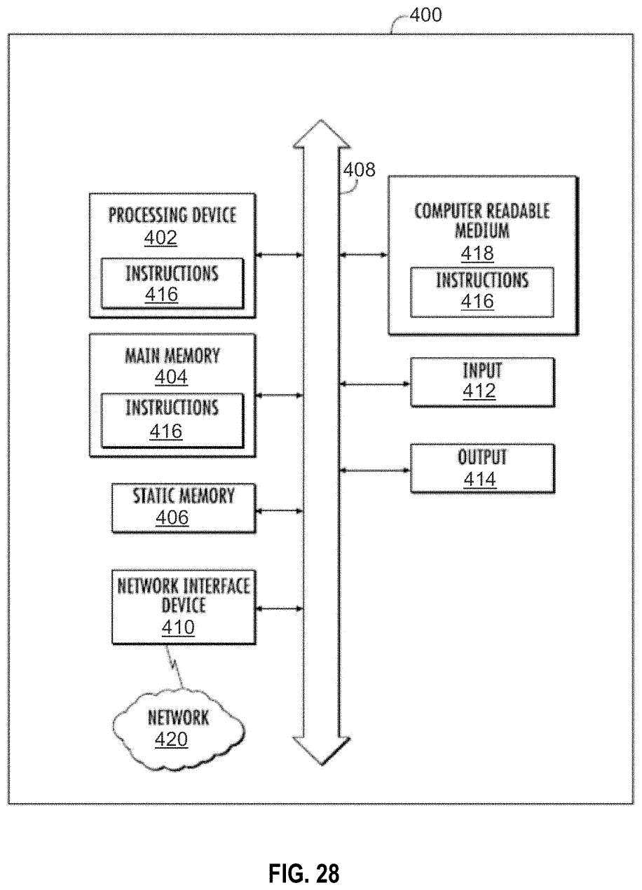

FIG. 28 is a schematic diagram of a generalized representation of a computer system 400 (optionally embodied in a computing device) that can be utilized as, or included in a component of, a control module as disclosed herein. In this regard, the computer system 400 is adapted to execute instructions from a computer-readable medium to perform these and/or any of the functions or processing described herein. The computer system 400 in FIG. 28 may include a set of instructions that may be executed to program and configure programmable digital circuits for controlling a system for mitigating airborne contamination of a conditioned indoor environment. The computer system 400 may be connected (e.g., networked) to other machines in a LAN, an intranet, an extranet, or the Internet. While only a single device is illustrated, the term “device” shall also be taken to include any collection of devices that individually or jointly execute a set (or multiple sets) of instructions to perform any one or more of the methodologies discussed herein. The computer system 400 may be a circuit or circuits included in an electronic board card, such as a printed circuit board (PCB), a server, a personal computer, a desktop computer, a laptop computer, a personal digital assistant (PDA), a computing pad, a mobile device, or any other device, and may represent, for example, a server or a user's computer.

The computer system 400 in this embodiment includes a processing device or processor 402, a main memory 404 (e.g., read-only memory (ROM), flash memory, dynamic random access memory (DRAM), such as synchronous DRAM (SDRAM), etc.), and a static memory 406 (e.g., flash memory, static random access memory (SRAM), etc.), which may communicate with each other via a data bus 408. Alternatively, the processing device 402 may be connected to the main memory 404 and/or static memory 406 directly or via some other connectivity means. The processing device 402 may be a controller, and the main memory 404 or static memory 406 may be any type of memory.

The processing device 402 represents one or more general-purpose processing devices, such as a microprocessor, central processing unit, or the like. More particularly, the processing device 402 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 402 is configured to execute processing logic in instructions for performing the operations and steps discussed herein.

The computer system 400 may further include a network interface device 410. The computer system 400 also may or may not include an input 412, configured to receive input and selections to be communicated to the computer system 400 when executing instructions. The computer system 400 also may or may not include an output 414, including but not limited to a display, a video display unit (e.g., a liquid crystal display (LCD) or a cathode ray tube (CRT)), an alphanumeric input device (e.g., a keyboard), and/or a cursor control device (e.g., a mouse).

The computer system 400 may or may not include a data storage device that includes instructions 416 stored in a computer readable medium 418. The instructions 416 may also reside, completely or at least partially, within the main memory 404 and/or within the processing device 402 during execution thereof by the computer system 400, the main memory 404 and the processing device 402 also constituting computer readable medium. The instructions 416 may further be transmitted or received over a network 420 via the network interface device 410.

While the computer readable medium 418 is shown in an embodiment to be a single medium, the term “computer-readable medium” should be taken to include a single medium or multiple media (e.g., a centralized or distributed database, and/or associated caches and servers) that store the one or more sets of instructions. The term “computer readable medium” shall also be taken to include any medium that is capable of storing, encoding, or carrying a set of instructions for execution by the processing device 402 and that cause the processing device 402 to perform any one or more of the methodologies of the embodiments disclosed herein. The term “computer readable medium” shall accordingly be taken to include, but not be limited to, solid-state memories, optical media, and magnetic media.

The embodiments disclosed herein include various steps. The steps of the embodiments disclosed herein may be executed or performed by hardware components or may be embodied in machine-executable instructions, which may be used to cause a general-purpose or special-purpose processor programmed with the instructions to perform the steps. Alternatively, the steps may be performed by a combination of hardware and software.

The embodiments disclosed herein may be provided as a computer program product, or software, that may include a machine-readable medium (or computer readable medium) having stored thereon instructions which may be used to program a computer system (or other electronic devices) to perform a process according to the embodiments disclosed herein. A machine-readable medium includes any mechanism for storing or transmitting information in a form readable by a machine (e.g., a computer). For example, a machine-readable medium includes: a machine-readable storage medium (e.g., ROM, random access memory (“RAM”), a magnetic disk storage medium, an optical storage medium, flash memory devices, etc.); and the like.

Unless specifically stated otherwise and as apparent from the previous discussion, it is appreciated that throughout the description, discussions utilizing terms such as “analyzing,” “processing,” “computing,” “determining,” “displaying,” or the like, refer to the action and processes of a computer system, or a similar electronic computing device, that manipulates and transforms data and memories represented as physical (electronic) quantities within registers of the computer system into other data similarly represented as physical quantities within the computer system memories or registers or other such information storage, transmission, or display devices.

The algorithms and displays presented herein are not inherently related to any particular computer or other apparatus. Various systems may be used with programs in accordance with the teachings herein, or it may prove convenient to construct more specialized apparatuses to perform the required method steps. The required structure for a variety of these systems is disclosed in the description above. In addition, the embodiments described herein are not described with reference to any particular programming language. It will be appreciated that a variety of programming languages may be used to implement the teachings of the embodiments as described herein.

Those of skill in the art will further appreciate that the various illustrative logical blocks, modules, circuits, and algorithms described in connection with the embodiments disclosed herein may be implemented as electronic hardware, instructions stored in memory or in another computer readable medium and executed by a processor or other processing device, or combinations of both. The components of the system described herein may be employed in any circuit, hardware component, integrated circuit (IC), or IC chip, as examples. Memory disclosed herein may be any type and size of memory and may be configured to store any type of information desired. To clearly illustrate this interchangeability, various illustrative components, blocks, modules, circuits, and steps have been described above generally in terms of their functionality. How such functionality is implemented depends on the particular application, design choices, and/or design constraints imposed on the overall system. Skilled artisans may implement the described functionality in varying ways for each particular application, but such implementation decisions should not be interpreted as causing a departure from the scope of the present embodiments.

The various illustrative logical blocks, modules, and circuits described in connection with the embodiments disclosed herein may be implemented or performed with a processor, a Digital Signal Processor (DSP), an Application Specific Integrated Circuit (ASIC), a Field Programmable Gate Array (FPGA), or other programmable logic device, a discrete gate or transistor logic, discrete hardware components, or any combination thereof designed to perform the functions described herein. Furthermore, a controller may be a processor. A processor may be a microprocessor, but in the alternative, the processor may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration).

The embodiments disclosed herein may be embodied in hardware and in instructions that are stored in hardware, and may reside, for example, in RAM, flash memory, ROM, Electrically Programmable ROM (EPROM), Electrically Erasable Programmable ROM (EEPROM), registers, a hard disk, a removable disk, a CD-ROM, or any other form of computer readable medium known in the art. A storage medium is coupled to the processor such that the processor can read information from, and write information to, the storage medium. In the alternative, the storage medium may be integral to the processor. The processor and the storage medium may reside in an ASIC. The ASIC may reside in a remote station. In the alternative, the processor and the storage medium may reside as discrete components in a remote station, base station, or server.

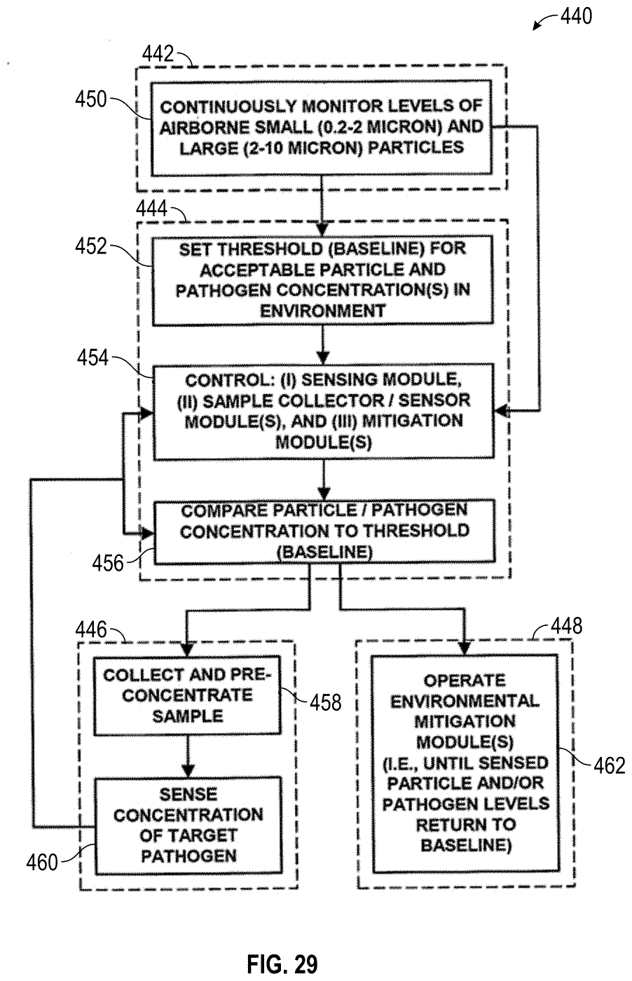

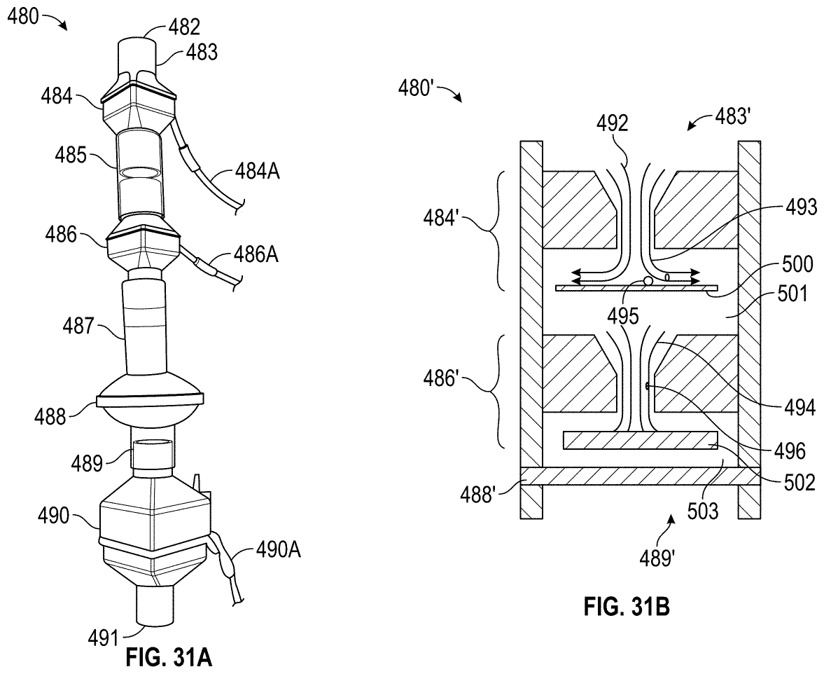

FIG. 29 is a schematic diagram showing components of a system 440 for detecting and mitigating airborne contamination with continuous monitoring and periodic sampling. The system 440 includes an airborne particle sensing module 442, an artificial intelligence (AI) application (or app) 444 that may embody a control module implemented in computer hardware and software (in one or more a stationary computing devices and/or mobile computing devices such as a smartphone or tablet computer), a sample collector and sensor module 446, and one or more environmental mitigation modules 448. According to block 450, the airborne particle sensing module 442 may be used for continuous monitoring of airborne concentrations of small particles (e.g., 0.2-2.0 μm diameter) and larger particles (e.g., 2.0-10 μm diameter). Signals from the airborne particle sensing module 442 may be supplied to the AI app 444. According to block 452, the AI app 444 may be used to set a threshold (e.g., baseline level) for acceptable particulate level and/or specific pathogen level in the environment being monitored. As noted in block 454, the AI app 444 may be used to control the sensing module 442, the sample collection/sensor module 446, and the environmental mitigation module 448. According to block 456, a concentration of particles detected by the airborne particle sensing module 442 is compared to a particulate concentration threshold or baseline. Additionally or alternatively according to block 456, a concentration of pathogens detected by the sample collection/sensor module 446 is compared to a pathogen concentration threshold or baseline. (The sample collection/sensor module 446 may serve to collect an air sample, preconcentrate one or more pathogens on a preconcentration surface, and sense one or more pathogens such as SARS-SoV-2 on the preconcentration surface, wherein the sample collection and sensor module 446 may include one or more impactors, such as described in connection with FIGS. 31A-31B.) The preconcentration function preconcentrates the aerosol particles in the air on a surface, which improves the sensitivity of the device and allows for viral antigen measurement in low concentration aerosols. The sensing function detects the presence of a pathogen-specific antigen (e.g., SARS-CoV-2 antigen) on the preconcentration surface, using a highly sensitive probe (e.g., a quantum dot measurement technique) combined with an aptamer that only binds to a target protein of the pathogen (e.g., SARS-CoV-2's N protein), giving the technique extremely high selectivity even to viral matter in the same family (e.g. SARS-CoV-1 and MERS). If the comparison step of block 456 shows that a detected particle level exceeds a particulate threshold or baseline concentration and/or that a detected pathogen level exceeds a pathogen threshold or baseline concentration, then such comparison(s) may trigger the initiation or continuation of air sampling and pre-concentration (according to block 458) followed by sensing of concentration of the target pathogen(s) (according to block 460). In certain embodiments, the target pathogen(s) may include SARS-CoV-2. Additionally or alternatively, if the comparison step of block 456 shows that a detected particle level exceeds a particulate threshold or baseline, then operation of the environmental mitigation module(s) 448 may be initiated or modified (according to block 462) until a particulate level (e.g., sensed by the sensing module 442) and/or pathogen level (e.g., sensed by the sampling and sensor module 446) returns to at or below the relevant baseline level(s). The environmental mitigation module 448 may embody any suitable type of mitigation module(s) disclosed herein, including but not limited to filtration, ozone and/or ultraviolet disinfection, dry scrubbing, wet scrubbing, and the like, which may be associated with a HVAC or ventilation apparatus of a particular structure or environment. In certain embodiments, the system 442 may be referred to as a Transmission Reduction Artificial Intelligence (AI) System or “TRAIS.”

In certain embodiments, one group of steps according to various blocks described above may be performed in an intermittent operational mode, while another group of steps according to various blocks described above may be performed in a continuous mode. For example, an intermittent operational mode may include performance of steps described in blocks 450, 454, 456, 458, 460, and 462 (optionally with block 452), while a continuous operational mode may include performance of steps described in blocks 442, 454, 456, and 462.

In certain embodiments, an ionic liquid/glycerol-based sensing platform allows the a pathogen sensor (e.g., SARS-CoV-2 sensor) to be free of evaporative considerations and provide a durable support for a sensing reaction. A custom-made aptamer whose configuration allows it to only bind to SARS-CoV-2's proteins and provides the selectivity for the robust detection of SARS-CoV-2 on a long-lasting probe. A quantum dot Förster resonance energy transfer (FRET) signal transduction mechanism allows for sensitive readout of viral signal in a single reaction step, free of washing and liquid handling operations. This robust design allows the device to eventually be used in a “plug-and-play” manner without significant procedural (e.g. calibration, reagent replacement) needs to increase reproducibility of SARS-CoV-2 measurements. If the system detects the presence of any SARS-CoV-2 viral antigen, then one or more mitigation modules (which may plug into or otherwise be installed within) a building ventilation control system may take appropriate action (e.g., carrying outdoor fresh air into the room and transporting high aerosol particle air that has been determined to contain pathogen to a disinfection system with a filter and UV light system). This system design conserves outstanding ventilation energy for aerosol mitigation at an estimated daily cost savings (e.g. 23-fold if system activates only once in 24 hours), allowing for data-driven approaches to reduce viral transmission in hospitals and other buildings without significant increases in ventilation energy consumption.

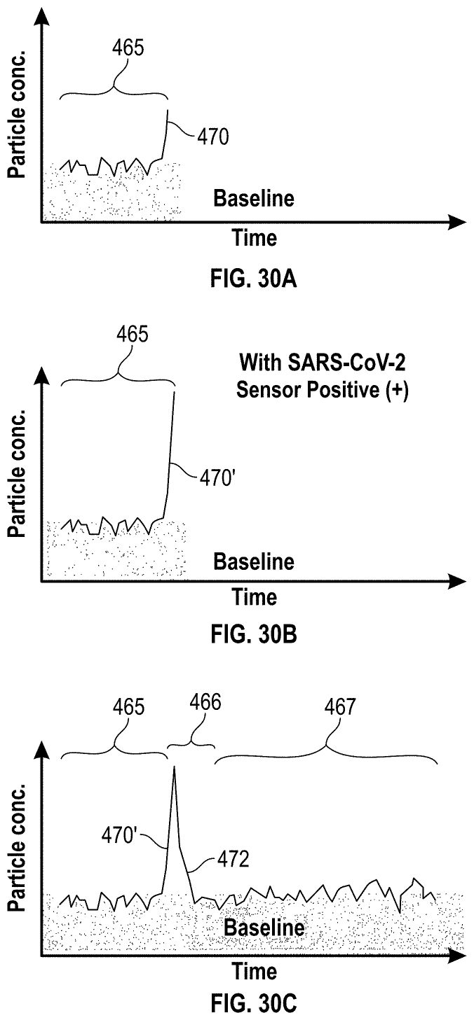

FIGS. 30A-30C are plots of particle concentration versus time illustrating performance of a method (for detecting and mitigating airborne contamination with continuous monitoring and periodic sampling) including steps identified in FIG. 29. In FIG. 30A, particulate levels in an environment are continuously monitored during a first time window 465. During a majority of the first time window 465, sensed particulate levels are within a baseline range, until an initial spike 470 is detected. Detection of the initial spike 470 triggers air sampling, preconcentration, and sensing of one or more target pathogens. As shown in FIG. 30B, the initial spike 470 may grow to a larger spike 470′ while the steps of air sampling, preconcentration, and sensing are performed to identify presence and/or concentration of one or more target pathogens such as SARS-CoV-2. Whether responsive to the initial spike 470 and/or the positive identification of one or more target pathogens, one or more mitigation modules may be operated to reduce concentration of particles and pathogens in the environment being monitored. FIG. 30C incorporates the plots of FIGS. 30A-30B, and shows the effect of operation of one or more mitigation modules. As shown, the first time window 465 is followed by a second time window 466 (in which particle concentration initially spikes according to spike 470′ but is returned (by declining spike 472) to within a baseline level through operation of one or more mitigation modules during a third time window 467.

FIG. 31A illustrates a first collector and sensor assembly 480 useable with the system of FIG. 29, including serially arranged first and second impactors 484, 486, a filter 488, and a fan 490, wherein the impactors 484, 486 may serve as pathogen sensors. The collector and sensor assembly 480 includes a first pipe section 483 arranged to conduct an air sample from an inlet 482 to the first impactor 484, which may be configured to sense larger aerosols (e.g., PM10, from 2.0 μm to 10.0 μm diameter. A second pipe section 485 is arranged downstream of the first impactor 484 and is configured to direct the air sample to a second impactor 486, which may be configured to sense smaller aerosols (e.g., PM2.5, or 0.2 μm-2.5 μm size range). A third pipe section 487 is arranged to direct the air sample to a viral/bacterial filter 488, which is arranged (together with a fourth pipe section 489) between the second impactor 486 and a fan 490 that generates subatmospheric pressure to draw an air sample through the collector and sensor assembly 480. An air outlet 491 is arranged downstream of the fan 490. The first impactor 484 and second impactor 486 have associated first and second impactor wires 484A, 486A, respectively, coupled to sensors (e.g., CMOS sensor) of the respective impactors 484, 486, while power signal wires 490A are arranged to conduct power from a power source (e.g., battery or AC outlet power, not shown) to the fan 490.

FIG. 31B is a cross-sectional view of a portion of a collector and sensor assembly 480′ similar to the assembly 480 depicted in FIG. 31A, showing serially arranged first and second impactors 484′, 486′ and a filter 488′. The first impactor 484′ receives an inlet air sample 492 from an upstream pipe 483′ and conveys it through a first nozzle to a chamber 501 of the first impactor 484′. The inlet air sample 492 is directed against and around a first preconcentrator/sensor surface 500 that may include a first CMOS sensor, wherein large aerosols or particles 495 may be captured by the first preconcentrator/sensor surface 500. A continued portion of the air sample 494 flows downstream to the second impactor 486, through a nozzle 494 to enter a chamber 503 and be directed against and around a second preconcentrator/sensor surface 502 that may include a second CMOS sensor, wherein smaller aerosols or particles 496 may be captured by the second preconcentrator/sensor surface 502. A further portion of the air sample then flows through the filter 488′ and a downstream pipe 489′ due to suction provided by a downstream fan (not shown).

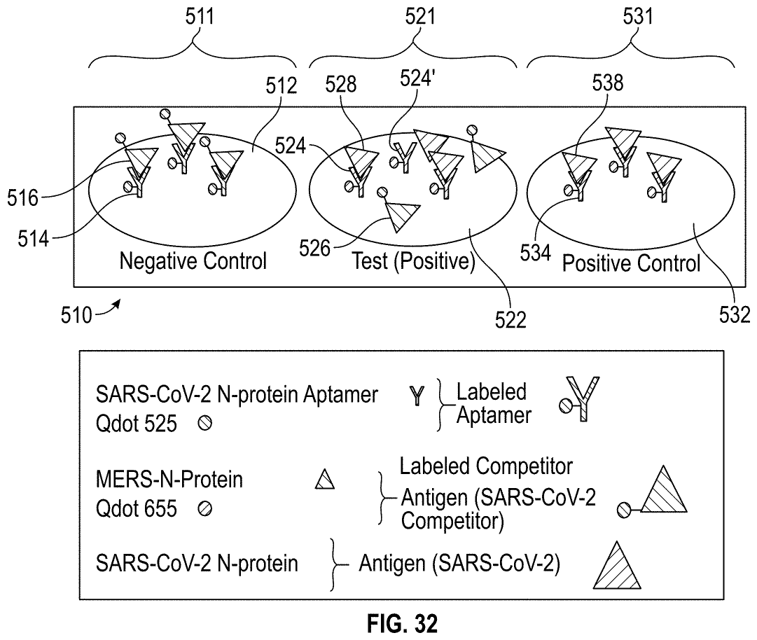

FIG. 32 schematically illustrates a sensor assembly 510 for detecting SARS-CoV-2 virus particles, including a negative control area 511, a positive control area 531, and a testing area 521. The negative control area 511 includes a substrate 512 having affixed thereto SARS-CoV-2 N-protein adaptamers labeled with 525 nm quantum dots (collectively, labeled adaptamers 514). As shown, MERS N-proteins labeled with 655 nm quantum dots (collectively, labeled competitor antigens) are bound to the labeled adaptamers 514 in the negative control area 511. The positive control area 531 includes a substrate 532 having labeled adaptamers 534 affixed thereto, wherein unlabeled SARS-CoV-2 N-protein antigens 538 are bound to the labeled adaptamers 534. The test area 521 includes a substrate 522 having labeled adaptamers 524 affixed thereto, wherein unlabeled SARS-CoV-2 N-protein antigens 528 are bound to the labeled adaptamers 524, and additional labeled competitor antigens 526 are present but not bound to the labeled adaptamers 524. The test area 522 may also include labeled adaptamers 524′ to which no molecules are bound. In operation, the negative and positive control areas 511, 531 may be insulated from a sample air flow with a transparent chamber and exposed to an impactor detector from the backside of a sensor supporting substrate.

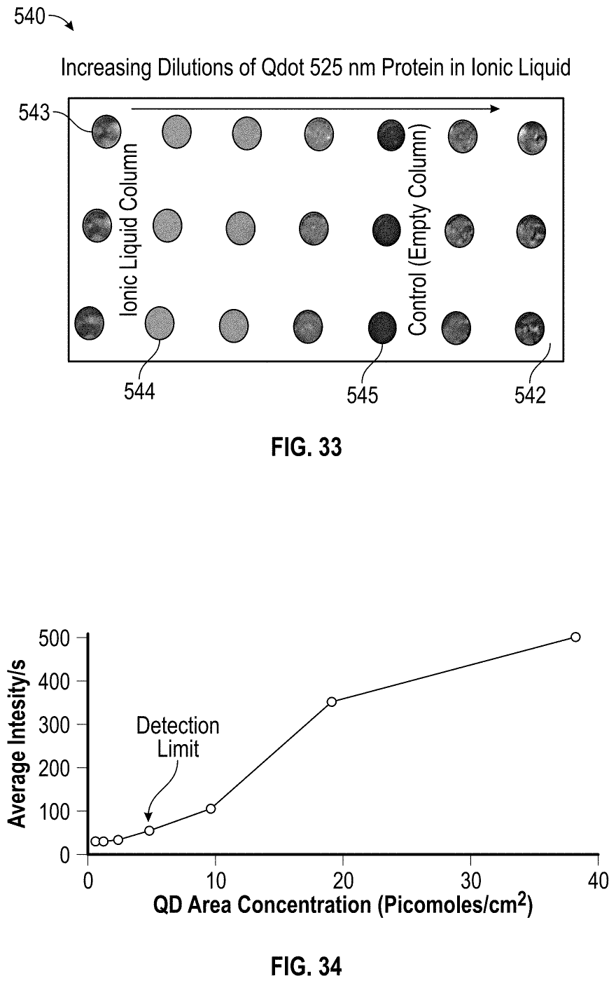

FIG. 33 illustrates a SARS-CoV-2 sensor image 540 obtained with a CMOS detector 542, with increasing dilutions of a 525 nm quantum dot labeled protein in ionic liquid. An ionic liquid column 543 is shown at right, a control (empty) column 545 is shown at middle left, and numerous sensing areas 544 show results of increasing dilutions of a 525 nm quantum dot labeled protein in ionic liquid. Green light intensity signals obtained by the CMOS detector 542 may be analyzed with custom software.

FIG. 34 is a calibration curve showing average intensity per exposure in seconds versus quantum dot surface area concentration (picomoles/cm²) in ionic liquid for the sensor image 540 of FIG. 33.

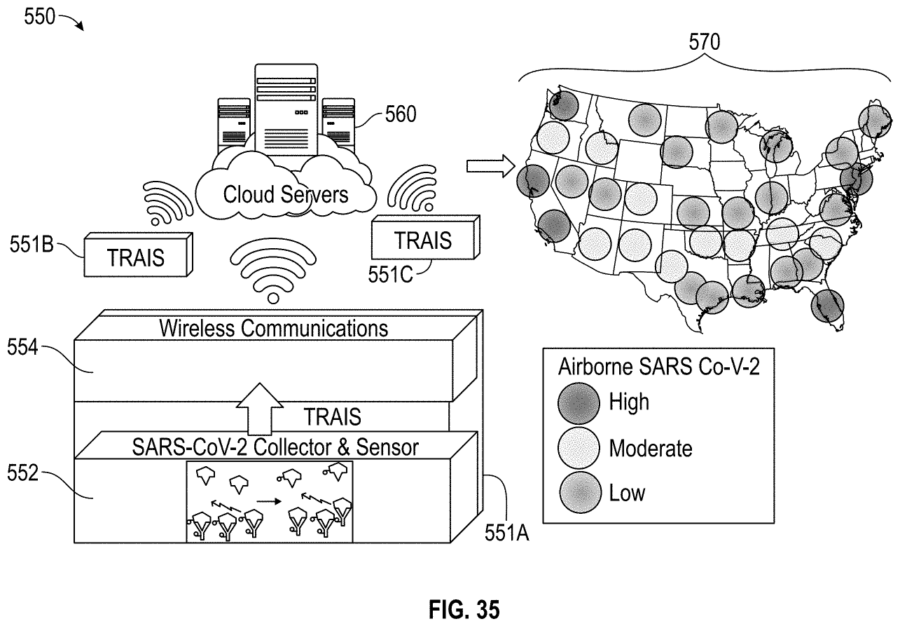

FIG. 35 schematically illustrates components of a wireless system 550 for communicating outputs of multiple collector and sensor assemblies (e.g., TRAIS) 551A-551C to one or more computer servers (e.g., cloud servers) 560 using one or more wireless and/or wired communication networks 554. Each TRAIS 551A-551C may include a pathogen collector and sensor module 552 and one or more wireless communication elements 554 (e.g., transceivers). Wireless communications via different mechanisms (e.g., Bluetooth®, ZigBee, WiFi, etc.) may be used. The cloud server allows for access by institutions that can utilize collected data for statistical analyses of SARS-CoV-2 transmission. Right) Representation of airborne SARS-CoV-2 map based on measurements by TRAIS can inform airborne spread of disease through the country. This can help inform public policy regarding prevention of transmission (e.g., business operations, mask wearing, etc.). As shown, measurement data uploaded to the one or more servers 560 may be used to generate an airborne pathogen (e.g., SARS-CoV-2) map 570. Representation of airborne pathogens (e.g., SARS-CoV-2) in a map (with geographic overlay) based on measurements by the pathogen collector and sensor modules 551A-551C can inform airborne spread of disease throughout a desired geographic area (e.g., a city, state, nation, or continent). The maps would enable determination of areas of high exposure that can be detrimental to health. Wireless communication can communicate levels of health threats via the cloud for analysis by epidemiologists. The data could be plotted at building level, street level, city level, county, state, regional and country level, thereby providing additional value to government and private organizations. Such data could help inform public policy regarding prevention of transmission (e.g., business operations, mask wearing, etc.).

It is noted that the operational steps described in any of the embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the embodiments may be combined. Those of skill in the art will also understand that information and signals may be represented using any of a variety of technologies and techniques. For example, data, instructions, commands, information, signals, bits, symbols, and chips, which may be referenced throughout the above description, may be represented by voltages, currents, electromagnetic waves, magnetic fields, particles, optical fields, or any combination thereof.

Those skilled in the art will appreciate that other modifications and variations can be made without departing from the spirit or scope of the invention.

Since modifications, combinations, sub-combinations, and variations of the disclosed embodiments incorporating the spirit and substance of the invention may occur to persons skilled in the art, the invention should be construed to include everything within the scope of the appended claims and their equivalents. The claims as set forth below are incorporated into and constitute part of this detailed description.

It will also be apparent to those skilled in the art that unless otherwise expressly stated, it is in no way intended that any method in this disclosure be construed as requiring that its steps be performed in a specific order. Accordingly, where a method claim below does not actually recite an order to be followed by its steps or it is not otherwise specifically stated in the claims or descriptions that the steps are to be limited to a specific order, it is no way intended that any particular order be inferred. Moreover, where a method claim below does not explicitly recite a step mentioned in the description above, it should not be assumed that the step is required by the claim.

### REFERENCES

1. Maclean, S. Causes and spread of infection. in Level 2 Diploma In Health & Social Care 424-448 (2014).
2. Stadnytskyi, V., Bax, C. E., Bax, A. & Anfinrud, P. The airborne lifetime of small speech droplets and their potential importance in SARS-CoV-2 transmission. Proc. Natl. Acad. Sci. 117,11875-11877 (2020).
3. Tran, K., Cimon, K., Severn, M., Pessoa-Silva, C. L. & Conly, J. Aerosol generating procedures and risk of transmission of acute respiratory infections to healthcare workers: a systematic review. PLoS One 7, (2012).
4. Liu, Y. et al. Aerodynamic analysis of SARS-CoV-2 in two Wuhan hospitals. Nature 1-4 (2020).
5. Baron, P. Generation and behavior of airborne particles (aerosols). (2010).
6. Howard, J. et al. Face masks against COVID-19: an evidence review. (2020).
7. Kaufman, A. Portable life support system. (1982).
8. Turak, N. Wearing a mask can significantly reduce coronavirus transmission, study on hamsters claims. (2020). Available at: https://www.cnbc.com/amp/2020/05/19/coronavirus-wearing-a-mask-can-reduce-transmission-by-75percent-new-study-claims.html?fbclid=lwAR1sSICZFohFW_bMW8_EFxqdvjY3GSGN9OrU3deMN2cyvYrEZ btHPPX0WEs. (Accessed: 10 Jun. 2020)
9. Santarpia, J. L. et al. Transmission potential of SARS-CoV-2 in viral shedding observed at the University of Nebraska Medical Center. MedRxIV (2020).
10. Zayas, G. et al. Cough aerosol in healthy participants: fundamental knowledge to optimize droplet-spread infectious respiratory disease management. BMC Pulm. Med. 12, 11 (2012).
11. Shaw, K. M. et al. Intensive care unit isolation hood decreases risk of aerosolization during noninvasive ventilation with COVID-19. Can. J. Anaesth. 1 (2020).
12. Simones, M. P. et al. Measurement of the size and charge distribution of sodium chloride particles generated by an Aeroneb Pro® pharmaceutical nebulizer. Eur. J. Nanomedicine 6, 29-36 (2014).
13. Procedural Safety Hood. (2020). Available at: https://bellmedical.com/stat-enclosure, 10 Jun. 2020
14. Forzani, E.; Tao, N. J.; Tsow, F., Method for assessing metabolic rate and maintaining indoor air quality and efficient ventilation energy use with passive environmental sensors. Patent filed in 2018.

1. A system for mitigating airborne contamination in a conditioned indoor environment, the system comprising:
at least one sensing module comprising an aerosol and/or particulate detector configured to detect presence and/or concentration of particles and/or aerosols at a location in the conditioned indoor environment;
at least one mitigation module configured to take one or more actions to reduce presence and/or concentration of particles and/or aerosols in the conditioned indoor environment; and
a control module employing an artificial intelligence algorithm configured to selectively activate the at least one mitigation module based on utilization of machine learning programmed rules and output signals from the at least one sensing module.

2. The system of claim 1, further comprising at least one sampling module configured to automatically collect an air sample from the conditioned indoor environment based on utilization of machine learning programmed rules and output signals from of the at least one sensing module.

3. The system of claim 2, wherein the at least one sampling module comprises at least one of a chemical analyzer or a biological analyzer configured to identify one or more constituents of the air sample.

4. The system of claim 1, wherein the at least one sensing module comprises at least one of a temperature sensor, a pressure sensor, a carbon dioxide sensor, a humidity sensor, and an occupancy sensor configured to sense the presence of at least one human within the conditioned indoor environment.

5. The system of claim 1, wherein the at least one mitigation module comprises a ventilation module configured to increase exchange of air between the conditioned indoor environment and an outdoor environment, wherein the ventilation module is configured to control an inlet fan and an outlet fan.

6. (canceled)

7. The system of claim 1, wherein the at least one mitigation module comprises at least one of the following items (i) to (vi): (i) a wet scrubber; (ii) a dry scrubber, (iii) a disinfection module configured to disinfect air of the conditioned indoor environment, (iv) an ozone generator, (v) an ultraviolet lamp, and (vi) a filtration module).

8. The system of claim 1, wherein the at least one mitigation module comprises a plurality of mitigation modules of different types.

9. The system of claim 1, wherein at least a portion of the at least one mitigation module is positioned in ductwork of an HVAC apparatus associated with the conditioned indoor environment.

10. The system of claim 1, further comprising a reporting module configured to receive at least one signal from the control module, and perform at least one of the following sequences (A) or (B):
(A) generate a user-perceptible alarm signal, or
(B) (i) receive at least one signal from the control module;
(ii) store information indicative of or derived from the at least one signal, and
(iii) generate one or more reports comprising information indicative of or derived from the at least one signal.

11. The system of claim 1, wherein the control module is further configured to control a HVAC apparatus associated with the conditioned indoor environment.

12. A method for mitigating airborne contamination in a conditioned indoor environment, the method comprising:
detecting presence and/or concentration of particles and/or aerosols at a location in the conditioned indoor environment using at least one sensing module that comprises an aerosol and/or particulate detector; and
utilizing a control module employing an artificial intelligence algorithm to selectively activate, based on utilization of machine learning programmed rules and output signals from the at least one sensing module, at least one mitigation module configured to take one or more actions to reduce presence and/or concentration of particles and/or aerosols in the conditioned indoor environment.

13. The method of claim 12, further comprising automatically collecting an air sample from the conditioned indoor environment, using at least one sampling module, based on utilization of machine learning programmed rules and output signals from the at least one sensing module.

14. The method of claim 13, further comprising identifying one or more constituents of the air sample using at least one of a chemical analyzer or a biological analyzer associated with the at least one sampling module.

15. The method of claim 12, wherein:
the at least one mitigation module comprises a ventilation module configured to increase exchange of air between the conditioned indoor environment and an outdoor environment, the ventilation module comprising an inlet fan and an outlet fan; and
activating the ventilation module comprises using the outlet fan to exhaust at least a portion of air received from the conditioned indoor environment to an external environment, and comprises using the inlet fan to draw air from the external environment to the conditioned indoor environment.

16. The method of claim 12, wherein the at least one mitigation module comprises a dry scrubber, and activating the dry scrubber comprises directing an air stream received from the conditioned indoor environment to contact one or more dry reagents configured to interact with constituents of the air stream.

17. The method of claim 12, wherein the at least one mitigation module comprises a wet scrubber, and activating the wet scrubber comprises directing an air stream received from the conditioned indoor environment to contact one or more liquid reagents configured to interact with constituents of the air stream.

18. The method of claim 12, wherein the at least one mitigation module comprises a disinfection module, and activating the disinfection module comprises activating at least one of an ozone generator or an ultraviolet lamp of the disinfection module.

19. The method of claim 12, wherein the at least one mitigation module comprises a filtration module that includes a filter and a diverter, and activating the filtration module comprises activating the diverter to direct an air stream received from the conditioned indoor environment to pass through the filter.

20. A non-transitory computer readable medium containing program instructions for receiving signals from at least one sensing modules and for controlling operation of at least one mitigation module configured to take one or more actions to reduce presence and/or concentration of particles and/or aerosols in a conditioned indoor environment, to perform a method comprising:
detecting presence and/or concentration of particles and/or aerosols at a location in the conditioned indoor environment using at least one sensing module that comprises an aerosol and/or particulate detector; and
utilizing a control module employing an artificial intelligence algorithm to selectively activate, based on utilization of machine learning programmed rules and output signals from the at least one sensing module, the at least one mitigation module configured to take one or more actions to reduce presence and/or concentration of particles and/or aerosols in the conditioned indoor environment.
